SEQUENCE LISTING (21 sequences)

SEQ ID NO: 1 - protein, 4 aa, synthetic construct
REDV

SEQ ID NO: 2 - protein, 4 aa, synthetic construct
DEGA

SEQ ID NO: 3 - protein, 5 aa, synthetic construct
YIGSR

SEQ ID NO: 4 - protein, 5 aa, synthetic construct
IKVAV

SEQ ID NO: 5 - protein, 5 aa, synthetic construct
YIGSR

SEQ ID NO: 6 - protein, 5 aa, synthetic construct
PHRSN

SEQ ID NO: 7 - protein, 5 aa, synthetic construct
PRARI

SEQ ID NO: 8 - protein, 4 aa, synthetic construct
CRGD

SEQ ID NO: 9 - protein, 4 aa, synthetic construct
RGDC

SEQ ID NO: 10 - protein, 5 aa, synthetic construct
CRGDS

SEQ ID NO: 11 - protein, 6 aa, synthetic construct
CRGDSG

SEQ ID NO: 12 - protein, 8 aa, synthetic construct
GPQGIWGQ

SEQ ID NO: 13 - protein, 8 aa, synthetic construct
GPQGIAGQ

SEQ ID NO: 14 - protein, 8 aa, synthetic construct
VPMSMRGG

SEQ ID NO: 15 - protein, 7 aa, synthetic construct
QPQGLAK

SEQ ID NO: 16 - protein, 8 aa, synthetic construct
GPLGLSGK

SEQ ID NO: 17 - protein, 5 aa, synthetic construct
PLGLA

SEQ ID NO: 18 - protein, 12 aa, synthetic construct
KCGPQGIWGQCK

SEQ ID NO: 19 - protein, 16 aa, synthetic construct
GKKCGPQGIWGQCKKG

SEQ ID NO: 20 - protein, 11 aa, synthetic construct
CGGPLGLAGGC

SEQ ID NO: 21 - protein, 16 aa, synthetic construct
GKKCGIQQWGGPCKKG

(19) United States
(12) Patent Application Publication
(10) Pub. No.: US 2025/0250397 A1
(43) Pub. Date: Aug. 7, 2025

(54) ANISOTROPIC POLY(ETHYLENE GLYCOL)-BASED HYDROGELS SYNTHESIZED VIA TWO-STAGE POLYMERIZATION AND MECHANICAL ALIGNMENT

(71) Applicant: University of Oregon, Eugene, OR (US)

(72) Inventors: Tayler Hebner, Eugene, OR (US); Danielle Benoit, Eugene, OR (US)

(73) Assignee: University of Oregon, Eugene, OR (US)

(21) Appl. No.: 19/043,299

(22) Filed: Jan. 31, 2025

Related U.S. Application Data
(60) Provisional application No. 63/548,744, filed on Feb. 1, 2024.

(51) Int. Cl. C08J 3/075; A61L 27/22; A61L 27/36; A61L 27/52; C08G 61/08; C08G 65/333; C08J 3/24

(52) U.S. Cl. CPC C08J 3/075; A61L 27/22; A61L 27/3687; A61L 27/52; C08G 61/08; C08G 65/33337; C08J 3/246; A61L 2430/10; C08G 2261/3324; C08G 2261/76

(57) ABSTRACT

Provided herein are anisotropic hydrogels, as well as methods and compositions for synthesizing anisotropic hydrogels, involving a two-stage polymerization and mechanical alignment. The anisotropic hydrogels may encapsulate cells, and can be used in vitro or in vivo for any purpose related to tissue engineering, and regenerative healing, e.g., of the musculoskeletal system.

### CROSS REFERENCE TO RELATED APPLICATION

This application claims the benefit of and priority to the earlier filing date of U.S. Provisional Application No. 63/548,744, filed Feb. 1, 2024, which is hereby incorporated by reference in its entirety.

### ACKNOWLEDGMENT OF GOVERNMENT SUPPORT

This invention was made with government support under grant number R01 AR064200 awarded by the National Institutes of Health. The government has certain rights in the invention.

### FIELD

The present disclosure generally relates to the field of tissue engineering and anisotropic hydrogel.

### INCORPORATION OF SEQUENCE LISTING

The Sequence Listing is submitted as an XML file in the form of the file named “1505-111483-02_Sequence Listing.xml” (˜19,188 bytes), which was created on Jan. 29, 2025 which is incorporated by reference herein.

### BACKGROUND

Fibrotic healing of surgically repaired tendon contributes to poor restoration of function and high rates of reinjury. The primary function of the tendon is to transmit forces from muscle to bone. When injured, force transmission is hindered, leading to chronic pain and disability. Tendon injuries pose a sizeable socioeconomic burden, with over 300,000 tendon repair procedures performed per year; over $400 million in medical costs are associated with flexor tendon injuries alone in the U.S. Despite efforts to repair injured tendons, restoration of function is rarely achieved, and re-injury is prominent. Nearly 40% of flexor tendon injuries heal with functional limitations, re-tearing occurs after up to 94% of rotator cuff repairs, and about 10% of repaired Achilles tendons re-rupture. The poor outcomes of tendon repair are attributed to fibrotic versus regenerative healing as cells bridge the gap between the sutured ends of the ruptured tendon. When a tendon heals, the tissue is not regenerated in the highly aligned hierarchical morphology characteristic of native, uninjured tendons. Instead, an inner-aligned extracellular matrix (ECM) bridge is formed across the injury site. The surrounding tissue consists of disorganized ECM. As force transmission relies on the mechanical properties of the anisotropic, organized tissue, inferior mechanical properties of the scar significantly impair tendon strength and range of motion.

Early, transient stages of healing are critical for orchestrating tissue regeneration versus fibrosis. Prior studies have defined the first 14 days after injury are critical to initiate tendon regeneration versus fibrotic scar tissue as cells bridge the injury site. While tenocytes are typically quiescent during homeostasis, it has been shown that fibroblast activation occurs between days 10 and 14 after injury, and elaboration of a new cell/matrix bridge is observed through day 12. Notably, these results show that the fibrotic cell populations associated with scar tissue persist in the fully healed tendon once formed within this transient healing phase. Therefore, any external intervention to suppress fibrotic scar tissue formation and promote regenerative healing is likely to be more effective within this initial transient healing phase when the healing milieu establishes cell orientation and phenotype.

### SUMMARY

Provided here are method of synthesizing an anisotropic hydrogel, comprising: reacting a multi-arm poly(ethylene glycol) (PEG) comprising maleimide end groups (PEG-maleimide), with a dithiol crosslinker and a monothiol molecule, thereby generating a first-stage crosslinked network with pendant thiol groups; applying mechanical force to stretch the first-stage crosslinked network; and reacting the pendant thiol groups with a multi-arm PEG comprising norbornene end groups (PEG-norbornene), by subjecting the first-stage crosslinked network to UV light in the presence of a photoinitiator, thereby generating the anisotropic hydrogel.

In some embodiments, the molar amount/concentration of the maleimide end groups to the molar amount/concentration of thiol groups in the dithiol crosslinker and the monothiol molecule is a ratio of about 20:23 (maleimide:thiol); the molar amount/concentration of monothiol molecule to the molar amount/concentration of dithiol crosslinker is a ratio of about 4:21 (monothiol molecule:dithiol crosslinker); and/or the molar amount/concentration of the pendant thiol groups to the molar amount/concentration of norbornene end groups is a ratio of about 1:1.

In some embodiments, the multi-arm PEG-maleimide comprises 3-arm, 4-arm, 6-arm, 8-arm, or 10-arm PEG, or any combination thereof; the multi-arm PEG-norbornene comprises 3-arm, 4-arm, 6-arm, 8-arm, or 10-arm PEG, or any combination thereof; the multi-arm PEG-maleimide has a molecular weight of about 2 to about 40 kDa, or any combination thereof; and/or the multi-arm PEG-norbornene has a molecular weight of about 2 to about 40 kDa, or any combination thereof.

In some embodiments, the dithiol crosslinker comprises a peptide having two cysteines; the monothiol molecule comprises a peptide having one cysteine; the dithiol crosslinker comprises a peptide that is 4-100 amino acids in length, or a combination thereof; and/or the monothiol molecule comprises a peptide that is 4-100 amino acids in length, or a combination thereof.

In some embodiments, the dithiol crosslinker comprises a peptide comprising a matrix metalloproteinase (MMP)-degradable sequence, and/or the monothiol molecule comprises an adhesive peptide. In some examples, the matrix metalloproteinase (MMP)-degradable sequence is GPQGIWGQ (SEQ ID NO: 12), and/or the adhesive peptide is CRGDSG (SEQ ID NO: 11).

In some embodiments, the multi-arm PEG-maleimide, the multi-arm PEG-norbornene, the dithiol crosslinker, the monothiol molecule, and the photoinitiator are present together in a mixture. In some embodiments, the concentration of the photoinitiator (wt %) to the total concentration (wt %) of the multi-arm PEG-maleimide, the multi-arm PEG-norbornene, the dithiol crosslinker, the monothiol molecule, and the photoinitiator in the mixture is a ratio of about 1:20 to about 1:80.

In some embodiments, the mixture further comprises cells. In some examples, the cells are from musculoskeletal tissues.

Also provided are anisotropic hydrogels synthesized by the methods of the present disclosure. In some aspects, the ratio of the elastic modulus of the hydrogel in a direction perpendicular to the applied force, to the elastic modulus of the hydrogel in a direction parallel to the applied force, is about 1:5 to about 2:3; and/or the hydrogel exhibits a Herman's orientation parameter of about 0.2 to about 0.9.

Also provided are anisotropic hydrogels, comprising a cross-linked network of a first multi-arm poly(ethylene glycol) (PEG), a second multi-arm PEG, a crosslinking peptide, and a masking peptide, wherein the crosslinking peptide links together two of the first multi-arm PEG, or links together one of the first multi-arm PEG and one of the second multi-arm PEG, wherein the link to the first multi-arm PEG comprises a thiosuccinimide group, and the link to the second multi-arm PEG comprises a thionorbornane group; and a portion of the PEG arms of the first multi-arm PEG are linked to the masking peptide through a thiosuccinimide group.

In some embodiments, the molar amount/concentration of PEG arms of the first multi-arm PEG that are linked to the masking peptide, to the molar amount/concentration of PEG arms of the first multi-arm PEG that are linked to the crosslinking peptide is a ratio of about 1:9; and/or the molar amount/concentration of the total thionorbornane, to the molar amount/concentration of the total thiosuccinimide linkage, is a ratio of about 3:20.

In some embodiments, the first multi-arm PEG comprises 3-arm, 4-arm, 6-arm, 8-arm, or 10-arm PEG, or any combination thereof; the second multi-arm PEG comprises 3-arm, 4-arm, 6-arm, 8-arm, or 10-arm PEG, or any combination thereof; the first multi-arm PEG has a molecular weight of about 2 to about 40 kDa, or any combination thereof; and/or the second multi-arm PEG has a molecular weight of about 2 to about 40 kDa, or any combination thereof.

In some embodiments, the crosslinking peptide comprises a peptide having two cysteines; the masking peptide comprises a peptide having one cysteine; the crosslinking peptide comprises a peptide that is 10-20 amino acid in length, or a combination thereof; and/or the masking peptide comprises a peptide that is 4-10 amino acid in length, or a combination thereof.

In some embodiments, the crosslinking peptide comprises a matrix metalloproteinase (MMP)-degradable sequence, and/or the masking peptide comprises an adhesive peptide.

In some embodiments, the anisotropic hydrogel further comprises cells.

In some embodiments, the ratio of the elastic modulus of the hydrogel in a direction perpendicular to the applied force, to the elastic modulus of the hydrogel in a direction parallel to the applied force, is about 1:5 to about 2:3; and/or the anisotropic hydrogel exhibits a Herman's orientation parameter of about 0.2 to about 0.9.

Also provided are compositions for generating an anisotropic hydrogel, comprising a multi-arm poly(ethylene glycol) (PEG) comprising maleimide end groups (PEG-maleimide), a multi-arm PEG comprising norbornene end groups (PEG-norbornene), a dithiol crosslinker, a monothiol molecule, and/or a photoinitiator.

The foregoing and other objects, features, and advantages of the invention will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: A diagram showing the healing process of tendon. In case of tendon rupture, surgery is typically performed to suture tendon stubs together, allowing the tendon to heal naturally. However, natural healing is a fibrotic process, and can lead to the formation of disorganized scar tissue, which does not perform like uninjured tendon. As a result, there is a limited restoration of tendon function, and re-injury is common. Methods for recapitulating anisotropy of native tendon are needed to promote regenerative healing.

FIGS. 2A-2E: Diagrams showing the process of synthesizing an exemplary anisotropic poly(ethylene glycol) (PEG)-based hydrogel with encapsulated cells. FIG. 2A shows the starting material. FIG. 2B shows the first-stage crosslinking, where maleimide (from the PEG-maleimide) reacts with thiol (from the monothiol and dithiol molecules) through thiol-Michael addition reaction. Mechanical strain is then applied to the first-stage crosslinked network (FIG. 2C). FIG. 2D shows the second-stage crosslinking, where norbornene (from the PEG-norbornene) reacts with remaining thiol (from the dithiol molecules) through thiol-ene photopolymerization. FIG. 2E shows that in the synthesized anisotropic hydrogel, tenocytes align with the direction of strain in the polymer network.

FIG. 3: Diffraction patterns of isotropic hydrogel (0% strain) and anisotropic hydrogel (300% strain).

FIG. 4: Plots of stress (Pa) over strain (%) for anisotropic hydrogels with strain applied along either the axis parallel (triangles) or perpendicular (circles) to the direction of polymer alignment (10% strain/min).

FIGS. 5A-5B: Alignment of human tenocytes within anisotropic hydrogels with non-degradable crosslinks. FIG. 5A shows the starting material for generating the anisotropic hydrogel, and an illustration indicating the orientation of tenocytes within the crosslinked network of the anisotropic hydrogel. FIG. 5B shows fluorescence microscopy images of the anisotropic hydrogel, and corresponding plots of tenocyte frequency, at days 1, 3, 7, and 14 after hydrogel synthesis. Alignment of tenocytes was monitored via fluorescence microscopy over 14 days (top row, DAPI in blue, rhodamine-phalloidin in pink, scale bars 500 μm) and quantified using OrientationJ in ImageJ (quantification performed on 2 mm×2 mm area of 24 stacked images spanning 800 μm), with the normalized frequency plotted as a function of angle relative to alignment direction. n=3; scale bars: 500 μm.

FIGS. 6A-6C: Alignment of human tenocytes within anisotropic or isotropic hydrogels with matrix metalloproteinase (MMP)-degradable crosslinks. FIG. 6A shows the starting material for generating the anisotropic hydrogel (or the control isotropic hydrogel, without strain application), and an illustration indicating the orientation of tenocytes within the crosslinked network of the anisotropic hydrogel. FIG. 6B shows fluorescence microscopy images of the anisotropic hydrogel, and corresponding plots of tenocyte frequency, at days 1, 3, 7, and 14 after hydrogel synthesis. FIG. 6C shows fluorescence microscopy images of the isotropic hydrogel and the anisotropic hydrogel, and corresponding plots of tenocyte frequency, at day 14 after synthesis. Alignment of tenocytes was monitored via fluorescence microscopy over 14 days (DAPI in blue, rhodamine-phalloidin in pink, scale bars 500 μm) and quantified using OrientationJ in ImageJ (quantification performed on 2 mm×2 mm area of 24 stacked images spanning 800 μm), with the normalized frequency plotted as a function of angle relative to alignment direction. n=3; scale bars: 500 μm.

FIGS. 7A-7B: Alignment of human tenocytes within anisotropic hydrogels with MMP-degradable crosslinks, non-degradable crosslinks, or 50% MMP-degradable crosslinks and 50% non-degradable crosslinks. FIG. 7A show microscopy images of the hydrogels at days 0, 3, and 7 after synthesis. FIG. 7B show corresponding plots of tenocyte frequency over their angle relative to alignment direction. Scale bars: 100 μm.

FIG. 8: A diagram showing in vivo use of anisotropic hydrogel. Anisotropic hydrogel eECM with encapsulated tenocytes will be implanted in the suture gap between tendon ends during repair of a ruptured mouse flexor tendon. The hydrogel will serve as a template for tenocytes bridging the suture gap during critical early stages of healing, and MMP-degradable crosslinks will enable tenocytes to replace the synthetic eECM with organized native matrix upon healing.

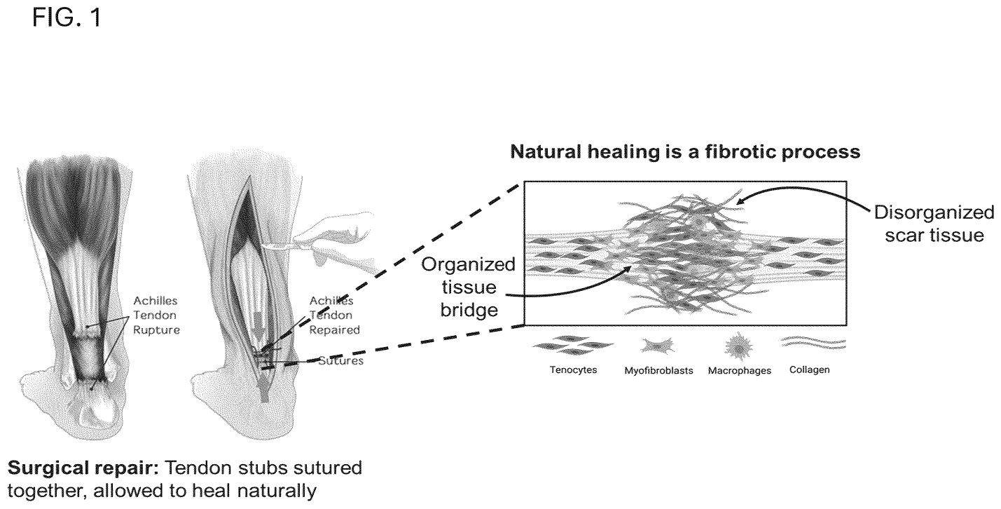

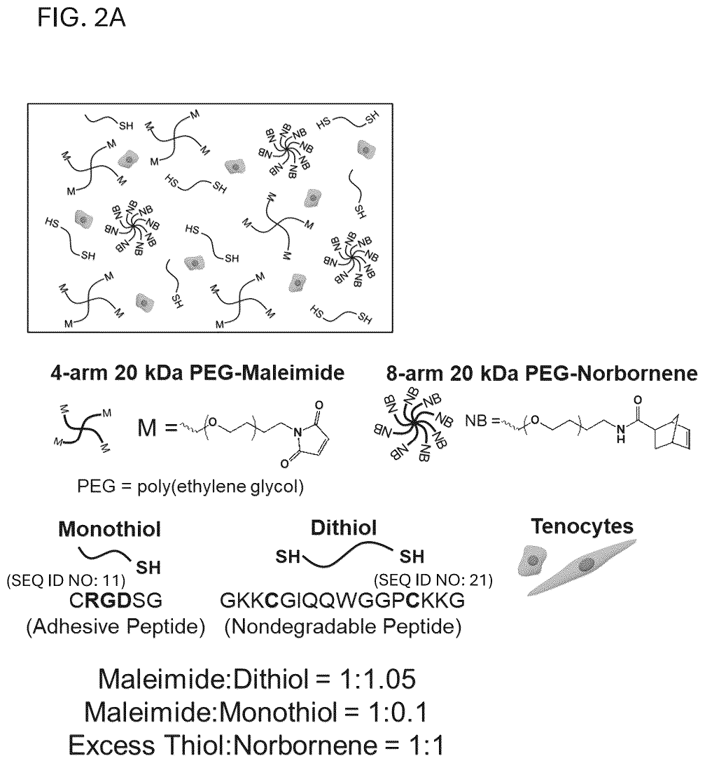

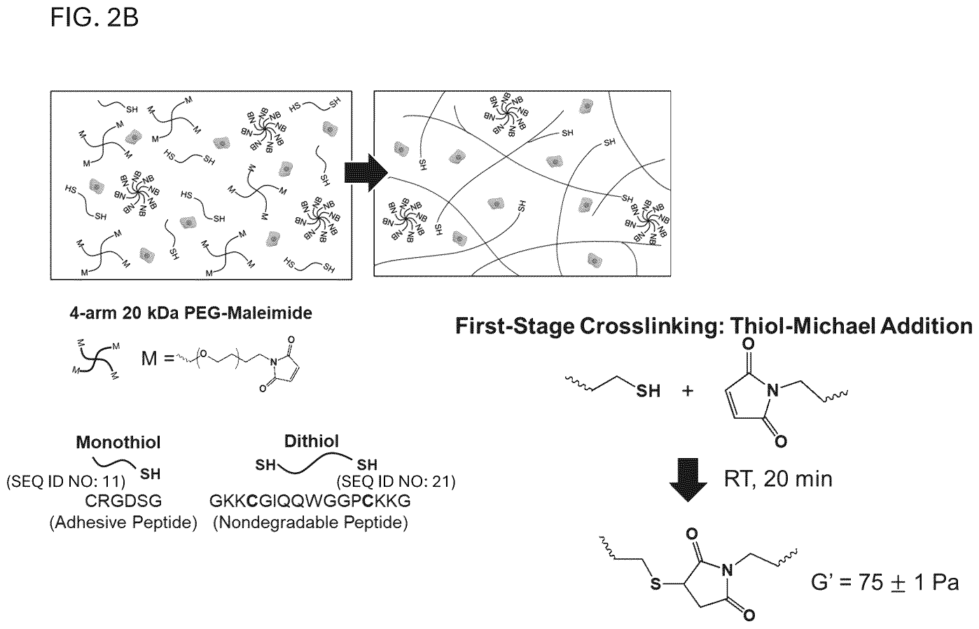

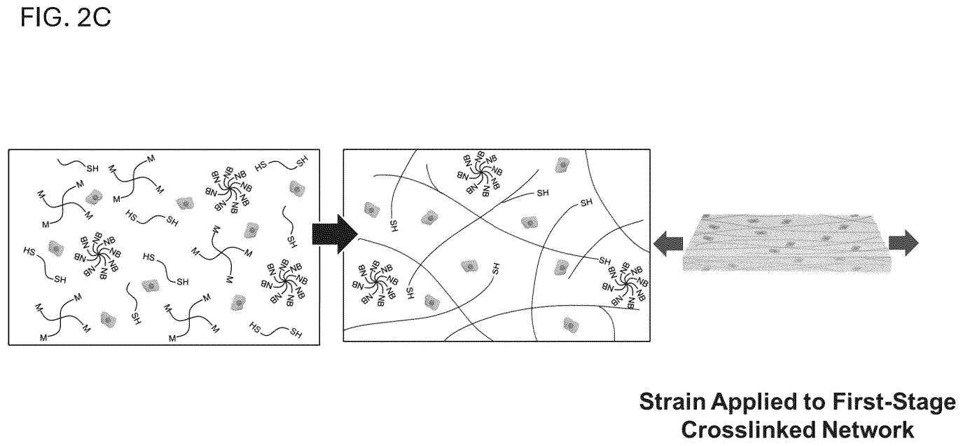

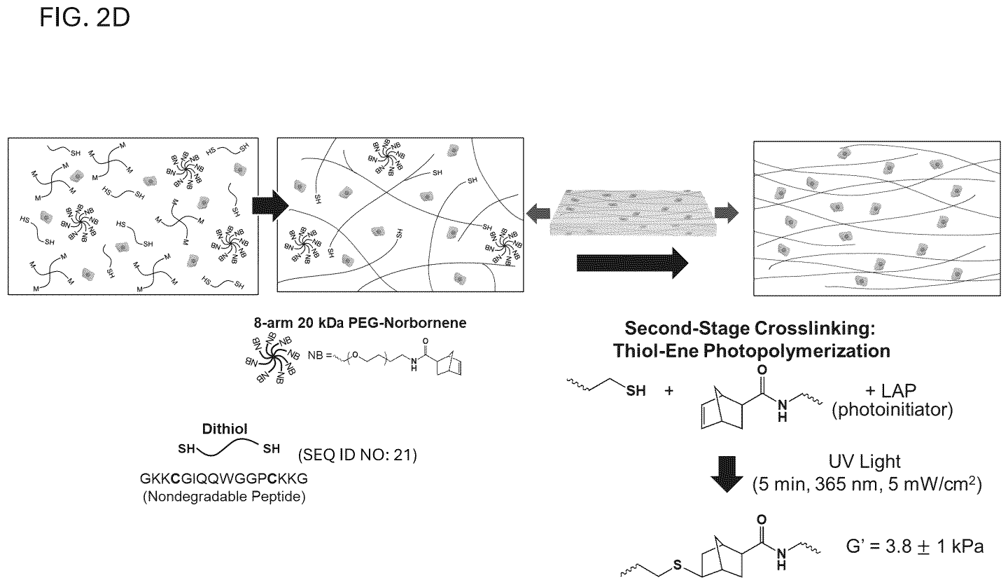

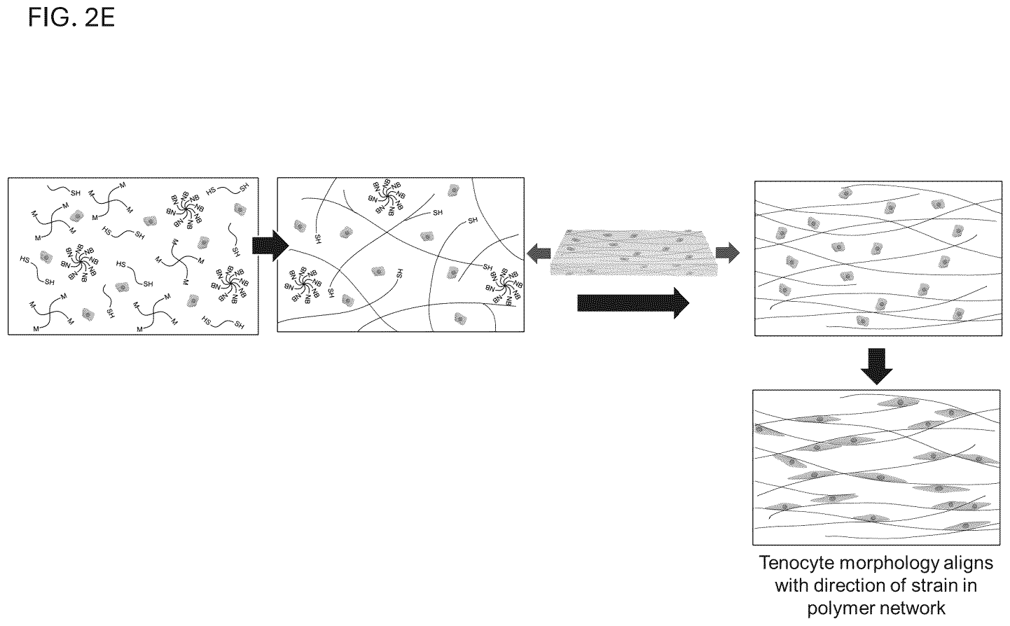

### DETAILED DESCRIPTION

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

### I. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “a cell” includes singular or plural cells and can be considered equivalent to the phrase “at least one cell.” As used herein, the term “comprises” means “includes.” For example, reference to “comprising a cell” includes one or a plurality of such cells. It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

In some examples, the numbers expressing quantities of ingredients, properties such as molecular weight, reaction conditions, and so forth, used to describe and claim certain aspects are to be understood as being modified in some instances by the term “about” or “approximately.” For example, “about” or “approximately” can indicate +/−10%, 5%, or 3% variation of the value it describes. Accordingly, in some aspects, the numerical parameters set forth herein are approximations that can vary depending upon the desired properties sought to be obtained by a particular embodiment. Notwithstanding that the numerical ranges and parameters setting forth the broad scope of some examples are approximations, the numerical values set forth in the specific examples are reported as precisely as practicable. The recitation of ranges of values herein is merely intended to serve as a shorthand method of referring individually to each separate value falling within the range.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein.

All molecules (e.g., proteins and nucleic acids) described herein, unless indicated otherwise, include any forms of the molecules, and include isolated, recombinantly produced, and manufactured molecules (e.g., by synthetic methods and recombinant technologies), and molecules in their natural environment.

To facilitate review of the various aspects, the following explanations of terms are provided:

Adhesive peptide: Peptides comprising a minimal motif required to specifically bind to a cell receptor responsible for cell adhesion (cell-adhesion motif). Cell adhesion motifs are known in the art. Exemplary adhesive motifs include RGD (arginine-glycine-aspartic acid), LDV, HAV, REDV (SEQ ID NO: 1), DEGA (SEQ ID NO: 2), YIGSR (SEQ ID NO: 3), IKVAV (SEQ ID NO: 4), YIGSR (SEQ ID NO: 5), PHRSN (SEQ ID NO: 6), and PRARI (SEQ ID NO: 7), etc. amino acid sequences. In some aspects, the adhesive peptide comprises one (or only one) cysteine. In some aspects, the adhesive peptide comprises RGD, which targets the integrin receptor. In some aspects, the adhesive peptide comprises RGD and one (or only one) cysteine (C). In some aspects, the adhesive peptide comprises CRGD (SEQ ID NO: 8). In some aspects, the adhesive peptide comprises RGDC (SEQ ID NO: 9). In some aspects, the adhesive peptide comprises CRGDS (SEQ ID NO: 10). In some aspects, the adhesive peptide comprises CRGDSG (SEQ ID NO: 11). In some aspects, the adhesive peptide is 4-100 amino acids in length, such as 3-12 amino acids in length, such as 3, 4, 5, 6, 7, 8, 9, 10, 11, or 12 amino acids in length. In some aspects, the adhesive peptide is 4-8 amino acids in length, such as 4, 5, 6, 7, or 8 amino acids in length.

Anisotropic: Having or exhibiting a physical property with different values when measured in different directions. Structural anisotropy of tissues in vivo arises from the preferred orientation of extracellular matrix proteins and/or cells, which imparts tissues with designated functions. Hydrogel anisotropy can be quantified by measuring the mechanical characteristics of materials, by image analysis, x-ray scattering, and mass transport characterization. In some aspects, mechanical characterization involves measurements of direction-specific (e.g., longitudinal vs. transverse) elastic moduli. The ratio of the mechanical characteristics along two different axes (generally, one in the direction parallel to the orientation of polymers, and the other one in the perpendicular direction) is used to determine the modulus anisotropy. Imaging of anisotropic hydrogels, e.g., with the use of electron microscopy or fluorescence microscopy, enables the visualization of a specific orientation. For example, the angles formed by cells with respect to a defined axis are quantified using a software, with a narrower distribution of angles indicating a higher degree of anisotropy. In some aspects, the hydrogels provided herein are anisotropic with respect to mechanical properties, such as elastic modulus.

Conservative variant: A peptide or protein containing conservative amino acid substitutions. Conservative amino acid substitutions are those substitutions that, when made, least interfere with the properties of the original peptide or protein, that is, the structure and especially the function of the original peptide or protein is conserved or not significantly changed by such substitutions.

Conservative amino acid substitution tables providing functionally similar amino acids are well known. The following six groups are examples of amino acids that are considered to be conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

In some aspects, a conservative variant of a peptide or protein provided herein can include at most 1, 2, 3, 4, 5, 6, or 7 conservative substitutions while retaining the properties of the original peptide (e.g., being cell-adhesive or MMP-degradable or non-MMP-degradable) at a desirable level (e.g., at least 70%, 75%, 80%, 85%, 90%, 95%, 98%, or 99% of the activity of the original peptide or protein) (also termed functional variant).

Dithiol crosslinker: A molecule comprising two thiol (—SH) groups, enabling it to participate in thiol-based crosslinking reactions, such as a thiol-ene reaction (e.g., Michael addition with acrylates or maleimides, free-radical additions, etc.). Either of the two thiol groups can be on a side chain or an end group of a polymeric chain. In some aspects, the dithiol crosslinker is a peptide comprising two cysteines. In some aspects, the dithiol crosslinker is an MMP-degradable peptide. In some aspects, the dithiol crosslinker is a non-MMP-degradable peptide. In some aspects, the peptide is 4-100 amino acids in length. In some aspects, the dithiol crosslinker is a polymeric chain (such as a PEG chain with 5-500 ethylene glycol units) terminated with thiol on both ends.

Gel: A colloidal system comprising a solid three-dimensional network within a liquid. By weight, a gel is primarily liquid, but behaves like a solid due to a three-dimensional network of entangled and/or crosslinked molecules of a solid within the liquid. From a rheological perspective, a gel has a storage modulus G′ value which exceeds that of the loss modulus G″. The storage modulus is a measure of the energy stored in a material in which a deformation (e.g., sinusoidal oscillatory shear) has been imposed; storage modulus can be thought of as the proportion of total rigidity of a material that is attributable to elastic deformation. The loss modulus is a measure of the energy dissipated in a material in which a deformation (e.g., sinusoidal oscillatory shear) has been imposed; loss modulus can be thought of as the proportion of the total rigidity of a material that is attributable to viscous flow rather than elastic deformation. The storage modulus and loss modulus can be determined with a rheometer.

Hydrogel: A three-dimensional crosslinked hydrophilic polymer. Hydrogels include a mixture of porous, permeable polymers and at least 10% by weight or volume of interstitial fluid (e.g., water). They can be highly absorbent yet maintain well defined structures. Hydrogels can be prepared using polymeric materials, including hyaluronic acid, poly(ethylene glycol), collagen, and gelatin. The hydrogels provided herein are anisotropic, and can include reversible and non-reversible covalent cross-linking bonds. In some examples, the hydrogels include cells.

Isolated: An “isolated” biological component, such as a nucleic acid, peptide, or protein, has been substantially separated or purified away from other biological components in the environment (such as a cell) in which the component occurs. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins.

Maleimide: A chemical group having a core structure

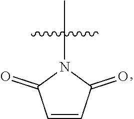 [SMILES: *N1C(=O)C=CC1=O]

wherein the core structure may comprise substituents bound to the ring carbon atoms.

Matrix metalloproteinase (MMP)-degradable: The property of being degradable or cleavable by a matrix metalloproteinase (MMP). MMPs are a family of calcium-dependent zinc-containing endopeptidases, including MMP-1, MMP-2, MMP-3, MMP-7, MMP-8, MMP-9, MMP-10, MMP-11, MMP-12, MMP-13, MMP-14, MMP-15, MMP-16, MMP-17, etc., which are responsible for tissue remodeling and degradation of extracellular matrix (ECM), including collagens, elastin, gelatin, matrix glycoproteins, and proteoglycan. The property of not being degradable or cleavable by an MMP is referred to as non-degradable or non-MMP-degradable. Exemplary MMP degradable amino acid sequences include GPQGIWGQ (SEQ ID NO: 12), GPQGIAGQ (SEQ ID NO: 13), VPMSMRGG (SEQ ID NO: 14), QPQGLAK (SEQ ID NO: 15), GPLGLSGK (SEQ ID NO: 16), PLGLA (SEQ ID NO: 17), etc.

Monothiol molecule: A molecule comprising one thiol group, enabling it to participate in thiol-based reactions, such as Michael addition with acrylates or maleimides. In some aspects, the monothiol molecule is a peptide comprising one cysteine. In some aspects, the monothiol molecule is an adhesive peptide. In some aspects, the peptide is 4-100 amino acids in length.

Multi-arm poly(ethylene glycol) (PEG): A branched polymer structure where more than one PEG chains (arms) are covalently attached to a central core molecule. Unlike linear PEG, which consists of a single chain, multi-arm PEGs have a star-like configuration with, e.g., 3, 4, 6, 8, 10 or more arms extending from the core. In some aspects, multi-arm PEGs are synthesized by ethoxylation of tripentaerythritol (8-arm PEG), hexaglycerol (8-arm PEG), dipentaerythritol (6-arm PEG), pentaerythritol (4-arm PEG), or glycerol (3-arm PEG). The number of ethylene oxide units in the PEG chain may not be equal for all arms. In some aspects, the multi-arm PEG can have a molecular weight of about 1 to about 100 kDa.

Musculoskeletal tissue: Tissues that make up the musculoskeletal system, which provides support, stability, movement, and protection to the body, including muscle tissues, skeletal tissues (bones and cartilage), connective tissues (ligaments, tendons, and fascia), and joints. Cells from musculoskeletal tissues are cells that found in, or isolated from these tissues (and optionally further grown or cultured in vitro), which may include tenocytes, ligamentocytes, osteoblasts, osteocytes, osteoclasts, bone lining cells, chondroblasts, chondrocytes, progenitor cells, skeletal muscle fibers, satellite cells, myoblasts, myosatellite cells, type A synoviocytes, type B synoviocytes, fibroblasts, mesenchymal stem cells, etc.

Norbornene: A chemical group having a core structure

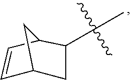 [SMILES: "," ; *C1CC2C=CC1C2]

wherein the core structure may comprise substituents bound to the ring carbon atoms.

Peptide: Used interchangeably with polypeptide and protein. A polymer in which the monomers are amino acid residues that are joined together through amide bonds. When the amino acids are alpha-amino acids, either the L-optical isomer or the D-optical isomer can be used, the L-isomers being preferred. The term as used herein encompasses any amino acid sequence and includes modified sequences such as glycosylated sequences. In some aspects, a peptide has an amino (N) terminus and a carboxy (C) terminus. In some aspects, the N- or C-terminus of a peptide can be modified, or joined to other molecules. In some aspects, a peptide can be linked to other molecules through its side chains.

Photoinitiator: A molecule that, when exposed to radiation (e.g., UV or visible), generates reactive species (e.g., free radicals, cations or anions), which initiates a polymerization reaction. A photoinitiator for thiol-ene reaction is a molecule that starts a thiol-ene reaction. Photoinitiators are well known in the art, including, lithium phenyl-2,4,6-trimethylbenzoylphosphinate (LAP), benzoin methyl ether (BME), camphorquinone (CQ), benzophenone (BP), thioxanthone (TX), benzoin isobutyl ether, diphenyl(2,4,6-trimethylbenzoyl) phosphine oxide, and 2,2-dimethoxy-2-phenyl acetophenone (DMPA).

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide or nucleic acid molecule will possess a relatively high degree of sequence identity when aligned using standard methods.

Methods of alignment of sequences for comparison are known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Variants of peptides provided herein can be characterized by possession of at least about 80%, for example at least about 85%, 90%, 95%, 96%, 97%, 98% or 99% sequence identity counted over the full-length alignment with the amino acid sequence of the original peptide using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). One of skill in the art will appreciate that these sequence identity ranges are provided for guidance only; it is entirely possible that variants with similar activity could be obtained that fall outside of the ranges provided.

Subject or patient: A term that includes human and non-human mammals. In some examples, the subject is a human or veterinary subject, such as a mouse, rat, dog, cat, or non-human primate. In some examples, the subject has a musculoskeletal injury, including damages to skeletal muscles, bones, tendons, joints, ligaments, and other affected soft tissues. In some examples, the subject has a tendon injury (such as a tendon rupture, tendon laceration, or tendinosis), a bone injury (such as a fracture, such as a non-union fracture, or due to cancer, osteoporosis, or osteoarthritis), or a wound.

Thioether link: A chemical bond where a sulfur atom connects two carbon atoms, or R—S—R′, wherein R and R′ are carbon-containing groups and can be independently aliphatic, cyclic aliphatic, or aromatic. A thioether link may be formed from a thiol-ene reaction, which is a reaction between a thiol and an alkene that forms a thioether, and may proceed through free-radical additions or Michael additions. In some examples, the alkene is from a norbornene. In some examples, the alkene is from a maleimide.

In some examples, the thioether link comprises Such a

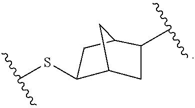 [SMILES: *SC1CC2CC1CC2* ; "."]

Such a link may also be refered to as a thionorbornane link herein.

In some examples, the thioether link comprises

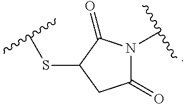 [SMILES: *SC1CC(=O)N(*)C1=O ; "."]

Such a link may also be refered to as a thiosuccinimide link herein.

### II. Materials

Provided herein are starting materials used to synthesize the anisotropic hydrogels disclosed herein, including multi-arm PEG-maleimide, multi-arm PEG-norbornene, monothiol molecules, dithiol crosslinkers, photoinitiators, and/or cells. Also provided are compositions comprising these materials for synthesizing anisotropic hydrogels.

### Multi-Arm PEG-Maleimide

Multi-arm PEG-maleimide refers to multi-arm PEG functionalized with one or more maleimide end groups. In some examples, one or more PEG arms of the multi-arm PEG-maleimide are linked to —(CH₂)ₙ—NH—C(═O)—(CH₂)ₘ—C₄H₂NO₂, wherein C₄H₂NO₂ represents the maleimide group, and n is 0 or any integer from 1 to 10, and m is 0 or any integer from 1 to 10. In some examples, one or more PEG arms of the multi-arm PEG-maleimide are linked to —(CH₂)ₙ—C₄H₂NO₂, wherein C₄H₂NO₂ represents the maleimide group, and n is any integer from 1 to 10. In some aspects, at least 50%, 60%, 70%, 75%, 80%, 85%, 90%, or 95%, or all of the PEG arms in the multi-arm PEG-maleimide are functionalized with a maleimide end group. In some aspects, the multi-arm PEG-maleimide comprises a 3-arm, 4-arm, 6-arm, 8-arm, or 10-arm PEG. In some aspects, the multi-arm PEG-maleimide comprises a glycerol core, a pentaerythritol core, a dipentaerythritol core, a tripentaerythritol core, or a hexaglycerol core. The number of ethylene oxide units in each PEG arm may or may not be equal for all arms, and in some examples may be independently from 2 to 1000. In some aspects, the multi-arm PEG-maleimide has a molecular weight of about 2 to about 40 kDa, such as about 2, 3, 4, 5, 6, 7, 8, 9, 10, 15, 20, 25, 30, 35, or 40 kDa. In some aspects, the multi-arm PEG-maleimide has a molecular weight of about 10 to about 30 kDa, or about 15 to about 25 kDa, such as about 15, 16, 17, 18, 19, 20, 21, 22, 23, 24, or 25 kDa. In some aspects, the multi-arm PEG-maleimide is a 4-arm PEG-maleimide comprising four maleimide end groups, and has a molecular weight of about 20 kDa. In some examples, the 4-arm PEG-maleimide comprises a pentaerythritol core.

In one example, the 4-arm PEG-maleimide is

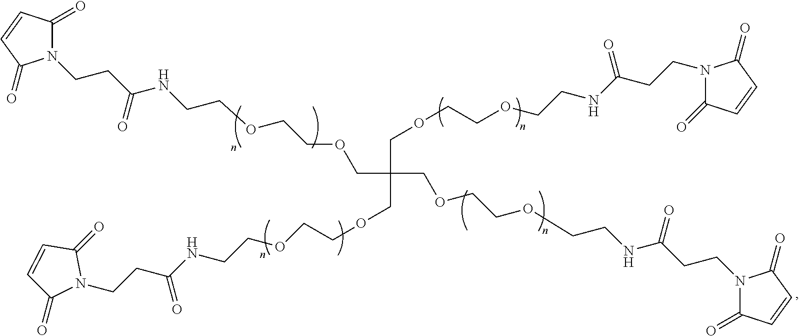

wherein each n is independently 2 to 1000.

Any combination of the above described multi-arm PEG-maleimide is also contemplated, such as at least 2, 3, 4, or 5 different multi-arm PEG-maleimide. For example, multi-arm PEG-maleimide can include at least 2, 3, 4, or 5 PEG-maleimide that have different arm numbers (such as 3, 4, 6, 8, 10, or any combination thereof), different core groups (such as a glycerol core, a pentacrythritol core, a dipentaerythritol core, a tripentaerythritol core, a hexaglycerol core, or any combination thereof), different levels of malcimide functionalization (functionalized arms/total arms, such as at least 50%, 60%, 70%, 75%, 80%, 85%, 90%, or 95%, or 100%, or any combination thereof), different molecular weights (such as any combination of any molecular weight from 2 to 40 kDa, such as 2, 3, 4, 5, 6, 7, 8, 9, 10, 11, 12, 13, 14, 15, 16, 17, 18, 19, 20, 21, 22, 23, 24, 25, 30, 35, or 40 kDa, or any combination thereof), different links to the maleimide group, etc., or any combination thereof.

### Multi-Arm PEG-Norbornene

Multi-arm PEG-norbornene refers to multi-arm PEG functionalized with one or more norbornene end groups. In some examples, one or more PEG arms of the multi-arm PEG-norbornene are linked to —(CH₂)ₙ—NH—C(═O)—C₇H₉, wherein C₇H₉ represents the norbornene group, and n is any integer from 1 to 10. In some examples, one or more PEG arms of the multi-arm PEG-norbornene are linked to —(CH₂)ₙ—O—C(═O)—C₇H₉, wherein C₇H₉ represents the norbornene group, and n is any integer from 1 to 10. In some examples, one or more PEG arms of the multi-arm PEG-norbornene are linked to —(CH₂)ₙ—C(═O)—NH—(CH₂) m-C₇H₉, wherein C₇H₉ represents the norbornene group, and n is any integer from 1 to 10, and m is 0 or any integer from 1 to 10. In some aspects, at least 50%, 60%, 70%, 75%, 80%, 85%, 90%, or 95%, or all of the PEG arms in the multi-arm PEG-norbornene are functionalized with a norbornene end group. In some aspects, the multi-arm PEG-norbornene comprises a 3-arm, 4-arm, 6-arm, 8-arm, or 10-arm PEG. In some aspects, the multi-arm PEG-norbornene comprises a glycerol core, a pentaerythritol core, a dipentaerythritol core, a tripentaerythritol core, or a hexaglycerol core. The number of ethylene oxide units in each PEG arm may or may not be equal for all arms, and in some aspects may be independently from 2 to 1000. In some aspects, the multi-arm PEG-norbornene has a molecular weight of about 2 to about 40 kDa, such as about 2, 3, 4, 5, 6, 7, 8, 9, 10, 15, 20, 25, 30, 35, or 40 kDa. In some aspects, the multi-arm PEG-norbornene has a molecular weight of about 10 to about 30 kDa, or about 15 to about 25 kDa, such as about 15, 16, 17, 18, 19, 20, 21, 22, 23, 24, or 25 kDa. In some aspects, the multi-arm PEG-norbornene is an 8-arm PEG-norbornene comprising eight norbornene end groups, and has a molecular weight of about 20 kDa. In some examples, the 8-arm PEG-norbornene comprises a tripentaerythritol core.

In one example, the 8-arm PEG-norbornene is

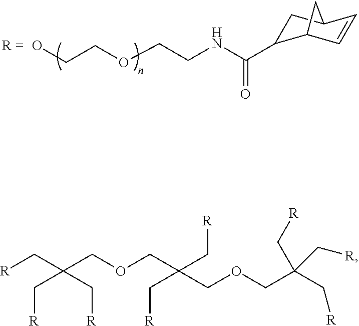

wherein each n is independently 2 to 1000.

Any combination of the above described multi-arm PEG-norbornene is also contemplated, such as at least 2, 3, 4, or 5 different multi-arm PEG-norbornene. For example, multi-arm PEG-norbornene can include at least 2, 3, 4, or 5 PEG-norbornene that have different arm numbers (such as 3, 4, 6, 8, 10, or any combination thereof), different core groups (such as a glycerol core, a pentaerythritol core, a dipentaerythritol core, a tripentaerythritol core, a hexaglycerol core, or any combination thereof), different levels of norbornene functionalization (functionalized arms/total arms, such as at least 50%, 60%, 70%, 75%, 80%, 85%, 90%, or 95%, or 100%, or any combination thereof), different molecular weights (such as any combination of any molecular weight from 2 to 40 kDa, such as 2, 3, 4, 5, 6, 7, 8, 9, 10, 11, 12, 13, 14, 15, 16, 17, 18, 19, 20, 21, 22, 23, 24, 25, 30, 35, or 40 kDa, or any combination thereof), different links to the norbornene group, etc., or any combination thereof.

### Monothiol Molecule

A monothiol molecule refers to a molecule comprising one thiol group, enabling it to participate in thiol-based reactions, such as a thiol-ene reaction. In some aspects, the monothiol molecule is a monothiol peptide.

In some embodiments, the peptide comprises one cysteine. In some embodiments, the peptide is an adhesive peptide. In some examples, the adhesive peptide comprises one or more amino acid sequences from RGD, LDV, HAV, any of SEQ ID NOs: 1-11, or any combination thereof. In some examples, the adhesive peptide comprises one or more functional variants of amino acid sequences from RGD, LDV, HAV, any of SEQ ID NOs: 1-11, or any combination thereof, which may include 1, 2, 3, 4, or 5 substitutions, such as conservative substitutions.

In some examples, the adhesive peptide comprises RGD. In some examples, the adhesive peptide comprises RGD and one cysteine. In some examples, the adhesive peptide comprises SEQ ID NO: 8. In some examples, the adhesive peptide comprises SEQ ID NO: 9. In some examples, the adhesive peptide comprises SEQ ID NO: 10. In some examples, the adhesive peptide comprises SEQ ID NO: 11. In some examples, the adhesive peptide comprises LDV, and comprises one cysteine, for example, at either end of LDV. In some examples, the adhesive peptide comprises HAV, and comprises one cysteine, for example, at either end of HAV. In some examples, the adhesive peptide comprises any of SEQ ID NOs: 1-7 (or a functional variant thereof), and comprises one cysteine, for example, at either end of any of SEQ ID NOs: 1-7 (or a functional variant thereof).

In some embodiments, the peptide is 4-100 amino acids in length, such as 4-90, 4-80, 4-70, 4-60, 4-50, 4-40, 4-30, 4-20, 4-10, 4-9, 5-90, 5-80, 5-70, 5-60, 5-50, 5-40, 5-30, 5-20, 5-10, 5-9, 6-90, 6-80, 6-70, 6-60, 6-50, 6-40, 6-30, 6-20, 6-10, or 6-9 amino acids in length. In some examples, the peptide is 3-12 amino acids in length, such as 3, 4, 5, 6, 7, 8, 9, 10, 11, or 12 amino acids in length. In some examples, the peptide is 4-8 amino acids in length, such as 4, 5, 6, 7, or 8 amino acids in length.

Any combination of the above described monothiol molecule is also contemplated, such as at least 2, 3, 4, or 5 different monothiol molecules. For example, the monothiol molecule can include at least 2, 3, 4, or 5 different adhesive peptides comprising different sequences and/or of different lengths.

### Dithiol Crosslinker

A dithiol crosslinker refers to a molecule comprising two thiol (—SH) groups, enabling it to participate in thiol-based crosslinking reactions, such as a thiol-ene reaction.

In some aspects, the dithiol crosslinker is a peptide. In some embodiments, the peptide comprises two cysteine residues. In some examples, the peptide comprises at least 5, 6, 7, 8, 9, 10, 11, or 12 amino acid residues between the two cysteine residues.

In some embodiments, the peptide is MMP-degradable. In some examples, the MMP-degradable peptide comprises any of SEQ ID NOs: 12-20; or a sequence having at least 85% identity to any of SEQ ID NOs: 12-20, and/or includes 1, 2, 3, 4, 5, 6, or 7 substitutions (e.g., conservative substitutions) compared to any of SEQ ID NOs: 12-20, while being MMP-degradable.

In some examples, the MMP-degradable peptide comprises SEQ ID NO: 12. In some examples, the MMP-degradable peptide comprises SEQ ID NO: 12, and comprises two cysteines, e.g., at both ends of SEQ ID NO: 12. In some examples, the MMP-degradable peptide comprises KCGPQGIWGQCK (SEQ ID NO: 18). In some examples, the MMP-degradable peptide comprises GKKCGPQGIWGQCKKG (SEQ ID NO: 19). In some examples, the MMP-degradable peptide comprises CGGPLGLAGGC (SEQ ID NO: 20). In some examples, the MMP-degradable peptide is degradable by MMP-1, MMP-2, MMP-3, MMP-9, MMP-13, and/or MMP-14. In some examples, the MMP-degradable peptide comprises SEQ ID NO: 13, and comprises two cysteines, e.g., at both ends of SEQ ID NO: 13. In some examples, the MMP-degradable peptide comprises SEQ ID NO: 14, and comprises two cysteines, e.g., at both ends of SEQ ID NO: 14. In some examples, the MMP-degradable peptide comprises SEQ ID NO: 15, and comprises two cysteines, e.g., at both ends of SEQ ID NO: 15. In some examples, the MMP-degradable peptide comprises SEQ ID NO: 16, and comprises two cysteines, e.g., at both ends of SEQ ID NO: 16. In some examples, the MMP-degradable peptide comprises SEQ ID NO: 17, and comprises two cysteines, e.g., at both ends of SEQ ID NO: 17.

In some embodiments, the peptide is non-MMP-degradable. In some examples, the non-MMP-degradable peptide comprises GKKCGIQQWGGPCKKG (SEQ ID NO: 21); or a sequence having at least 85% identity to SEQ ID NO: 21, and/or includes 1, 2, 3, 4, 5, 6, or 7 substitutions (e.g., conservative substitutions) compared to SEQ ID NO: 21, while being MMP-non-degradable.

In some embodiments, the peptide is a combination of MMP-degradable and non-MMP-degradable peptides, e.g., at a molar concentration ratio of about 1:1 to about 100:1 (degradable: non-degradable), such as about 1:1 to about 50:1, about 1:1 to about 40:1, about 1:1 to about 30:1, about 1:1 to about 20:1, about 1:1 to about 15:1, about 1:1 to about 10:1, or about 1:1 to about 5:1.

In some embodiments, the peptide is at most 100, 90, 80, 70, 60, 50, 40, 30, 20, or 18 amino acid in length, and/or at least 4, 5, 6, 7, 8, 9, 10, 11, 12, 13, 14, 15 amino acids in length. In some examples, the peptide is 4-100, 4-90, 4-80, 4-70, 4-60, 4-50, 4-40, 4-30, 4-20, 6-100, 6-90, 6-80, 6-70, 6-60, 6-50, 6-40, 6-30, 6-20, 8-100, 8-90, 8-80, 8-70, 8-60, 8-50, 8-40, 8-30, 8-20, 10-100, 10-90, 10-80, 10-70, 10-60, 10-50, 10-40, 10-30, or 10-20 amino acids in length, such as 5, 6, 7, 8, 9, 10, 11, 12, 13, 14, 15, 16, 17, 18, 19, 20, 21, 22, 23, 24, or 25 amino acids in length.

In some aspects, the dithiol crosslinker is a polymeric chain terminated with thiol on both ends and is non-MMP-degradable. In some embodiments, the dithiol crosslinker is a dithiol PEG. In some examples, the dithiol PEG is a linear PEG chain with about 5 to about 500 (e.g., about 5 to about 400, about 5 to about 300, about 5 to about 200, about 5 to about 100, about 10 to about 500, about 10 to about 400, about 10 to about 300, about 10 to about 200, about 10 to about 100, about 20 to about 500, about 20 to about 400, about 20 to about 300, about 20 to about 200, about 20 to about 100, about 30 to about 500, about 30 to about 400, about 30 to about 300, about 30 to about 200, or about 30 to about 100) ethylene glycol units terminated with thiol on both ends.

In some aspects, the dithiol crosslinker is a combination of dithiol MMP-degradable peptide, and dithiol PEG, e.g., at a molar concentration ratio of about 1:1 to about 100:1 (dithiol peptide: dithiol PEG), such as about 1:1 to about 50:1, about 1:1 to about 40:1, about 1:1 to about 30:1, about 1:1 to about 20:1, about 1:1 to about 15:1, about 1:1 to about 10:1, or about 1:1 to about 5:1.

Any combination of the above described dithiol crosslinker is also contemplated, such as at least 2, 3, 4, or 5 different dithiol crosslinkers. For example, the dithiol crosslinker can include at least 2, 3, 4, or 5 different dithiol crosslinkers comprising different sequences and/or of different lengths.

### Photoinitiator

A photoinitiator refers to a molecule that, when exposed to radiation (e.g., UV or visible light), generates reactive species (e.g., free radicals, cations or anions), which initiates a polymerization reaction. In some aspects, the photoinitiator initiates a thiol-ene reaction. In some aspects, the photoinitiator initiates a thiol-ene reaction in the presence of UV light. Any photoinitiator or any combination of photoinitiators that is suitable for initiating an intended reaction described herein is contemplated. Exemplary photoinitiators include lithium phenyl-2,4,6-trimethylbenzoylphosphinate (LAP), benzoin methyl ether (BME), camphorquinone (CQ), benzophenone (BP), thioxanthone (TX), benzoin isobutyl ether, diphenyl(2,4,6-trimethylbenzoyl) phosphine oxide, and 2,2-dimethoxy-2-phenyl acetophenone (DMPA). In one aspect, the photoinitiator is lithium phenyl-2,4,6-trimethylbenzoylphosphinate (LAP).

### Cells

Cells that can be encapsulated into the anisotropic hydrogel described herein can be any cell of interest, from any organism or subject, e.g., human and mammals. In some aspects, the cells are from musculoskeletal tissues. In some embodiments, the cells include tenocytes, ligamentocytes, tenoblasts, tendon stem/progenitor cells, osteoblasts, osteocytes, osteoclasts, bone lining cells, chondroblasts, chondrocytes, skeletal stem/progenitor cells, skeletal muscle cells (myocytes), skeletal muscle fibers, satellite cells, myoblasts, myosatellite cells, type A synoviocytes, type B synoviocytes, fibroblasts, mesenchymal stem cells, macrophages, neutrophils, etc., or any combination thereof.

### III. Methods and Compositions for Synthesizing Anisotropic Hydrogels

Provided herein are methods of synthesizing an anisotropic hydrogel, comprising: (i) reacting a multi-arm PEG-maleimide, with a dithiol crosslinker and a monothiol molecule, thereby generating a first-stage crosslinked network with pendant thiol groups; (ii) applying mechanical force to stretch the first-stage crosslinked network; and (iii) reacting the pendant thiol groups with a multi-arm PEG-norbornene, thereby generating the anisotropic hydrogel.

In step (i), the molar amount/concentration of the maleimide functional groups is in excess of the molar amount/concentration of the free thiol groups (the total amount/concentration from both the dithiol crosslinker and the monothiol molecule), so as to form a first-stage crosslinked network with pendant thiol groups. In some aspects, the molar amount/concentration of the maleimide groups to the molar amount/concentration of thiol groups (in the dithiol crosslinker and the monothiol molecule) is a ratio (e.g., mole ratio) of about 20:21 to about 20:30 (maleimide:thiol). In some examples, the ratio is about 20:21 to about 20:25 (maleimide:thiol). In one example, the ratio is about 20:23 (maleimide:thiol).

In some aspects, the molar amount/concentration of the monothiol molecule to the molar amount/concentration of the dithiol crosslinker is a ratio (e.g., mole ratio) of about 1:21 to about 8:21 (monothiol molecule: dithiol crosslinker). In some examples, the ratio is about 2:21 to about 6:21 (monothiol molecule: dithiol crosslinker). In some examples, the ratio is about 4:21 (monothiol molecule: dithiol crosslinker).

In some aspects, the molar amount/concentration of the pendant thiol groups to the molar amount/concentration of norbornene functional groups is a ratio (e.g., mole ratio) of about 1:1.

In some aspects, the reaction in step (i) is initiated by mixing the multi-arm PEG-maleimide with the dithiol crosslinker and the monothiol molecule. In some aspects, the reaction time is about 10 to about 120 minutes, such as about 10 to about 90 minutes, about 10 to about 60 minutes, about 10 to about 40 minutes, about 15 to about 35 minutes, about 15 to about 25 minutes, or about 20 minutes. In some aspects, the reaction temperature is room temperature. In some aspects, the reaction proceeds through the Michael addition mechanism, wherein maleimide groups react with thiol groups in a 1:1 stoichiometry to form thioether links (e.g., thiosuccinimide). In some aspects, all of the thiol groups from the monothiol molecule have reacted with the maleimide groups. In some aspects, a hydrogel is formed after the reaction, which includes the first-stage crosslinked network, water, and other reactants.

In step (ii), the first-stage crosslinked network (or hydrogel) is stretched, so as to orient the polymer chains along the stretching direction. Any suitable method to mechanically stretch the first-stage crosslinked network (or hydrogel) is contemplated. In some aspects, the stretch is applied unidirectionally or uniaxially. In some aspects, the first-stage crosslinked network (or hydrogel) is stretched to about 10% to about 500% strain, such as about 50% to about 450%, about 50% to about 400%, about 50% to about 350%, about 50% to about 300%, about 100% to about 450%, about 100% to about 400%, about 100% to about 350%, about 1000% to about 300%, about 150% to about 450%, about 150% to about 400%, about 150% to about 350%, about 150% to about 300%, about 100%, 200%, 300%, 400%, or 500% strain.

In step (iii), the free thiol groups (e.g., pendant thiol groups) remaining after the reaction in (i) are reacted (e.g., crosslinked) with a multi-arm PEG-norbornene, thereby generating the anisotropic hydrogel. In some aspects, this reaction is initiated by a photoinitiator in the presence of UV light. In some aspects, step (iii) is performed while retaining the applied strain from step (ii). In some aspects, the reaction or UV light exposure time is about 1 to about 30 minutes, such as about 1 to about 25 minutes, about 1 to about 20 minutes, about 1 to about 15 minutes, about 1 to about 10 minutes, about 1 to about 5 minutes, about 2 to about 30 minutes, about 2 to about 25 minutes, about 2 to about 20 minutes, about 2 to about 15 minutes, about 2 to about 10 minutes, or about 2 to about 5 minutes, or about 3, 4, 5, 6, 7, 8, 9, or 10 minutes. In some aspects, the reaction proceeds through a thiol-ene mechanism (e.g., initiated by free radical), wherein the norbornene groups of the multi-arm PEG-NB react with the pendant thiols on the first-stage network in a 1:1 stoichiometry to form thioether links (e.g., thionorbornane).

In some aspects, the multi-arm PEG-maleimide, the multi-arm PEG-norbornene, the dithiol crosslinker, the monothiol molecule, and the photoinitiator are present together in a mixture. In some aspects, all starting materials were combined in a buffer solution (e.g. phosphate buffered saline (PBS)) at an overall concentration of about 1 to about 20 wt %, such as about 1 to about 15 wt %, about 1 to about 10 wt %, about 1 to about 5 wt %, about 3 to about 20 wt %, about 3 to about 15 wt %, about 3 to about 10 wt %, about 3 to about 5 wt %, about 5 to about 20 wt %, about 5 to about 15 wt %, about 5 to about 10 wt %, or about 5 wt %. In some aspects, the multi-arm PEG-maleimide, the multi-arm PEG-norbornene, and the photoinitiator are first combined in a solution, e.g., to ensure homogenous mixing, before adding the dithiol crosslinker and the monothiol molecule to initiate the first-stage crosslinking. In some aspects, the photoinitiator is included in the mixture at a concentration of about 0.01 to about 1 wt %, such as about 0.05, 0.1, 0.2, 0.3, 0.4, or 0.5 wt % to initiate the photopolymerization. In some aspects, the concentration of the photoinitiator (wt %) to the total concentration (wt %) of the multi-arm PEG-maleimide, the multi-arm PEG-norbornene, the dithiol crosslinker, the monothiol molecule, and the photoinitiator in the mixture is a ratio of about 1:20 to about 1:80, such as about 1:30, 1:40, 1:50, 1:60, or 1:70.

In some aspects, the starting materials further comprises cells. In some aspects, the cells are present in the starting mixture at any desirable concentration, e.g., anywhere between 1×10⁴ to 1×10⁸ cells/mL, such as 1×10⁵ to 1×10⁷ cells/mL, such as about 1×10⁶ cells/mL. When cells are included in the starting materials, the synthesized anisotropic hydrogel encapsulates the cell. In some aspects, the cells are aligned in accordance with the direction of the aligned polymer.

Also provided are compositions for generating an anisotropic hydrogel, comprising a multi-arm PEG-maleimide, a multi-arm PEG-norbornene, a dithiol crosslinker, a monothiol molecule, and/or a photoinitiator. In some aspects, the compositions comprise the components at a ratio as described in the present methods for generating anisotropic hydrogels.

For example, in some aspects, the molar amount/concentration of the maleimide end groups to the molar amount/concentration of thiol groups in the dithiol crosslinker and the monothiol molecule in the composition is a ratio of about 20:23 (maleimide:thiol). In some aspects, the molar amount/concentration of monothiol molecule to the molar amount/concentration of dithiol crosslinker in the composition is a ratio of about 4:21 (monothiol molecule: dithiol crosslinker). In some aspects, the molar amount/concentration of the norbornene end groups to the molar amount/concentration of the maleimide end groups in the composition is a mole ratio of about 3:20.

### IV. Anisotropic Hydrogels

Provided herein are anisotropic hydrogels, comprising a cross-linked network of a first multi-arm PEG, a second multi-arm PEG, a crosslinking peptide, and a masking peptide. The first multi-arm PEG corresponds to the multi-arm PEG-maleimide reactant disclosed herein; the second multi-arm PEG corresponds to the multi-arm PEG-norbornene reactant disclosed herein; the crosslinking peptide corresponds to the dithiol peptide reactant disclosed herein, and the masking peptide corresponds to the monothiol peptide reactant disclosed herein.

In some aspects, the first multi-arm PEG, second multi-arm PEG, crosslinking peptide, and masking peptide are crosslinked together via thioether groups. In some examples, the links to the first multi-arm PEG comprise a thiosuccinimide group, and/or the links to the second multi-arm PEG comprises a thionorbornane group. In some examples, the crosslinking peptide links together two of the first multi-arm PEG, or links together one of the first multi-arm PEG and one of the second multi-arm PEG, wherein the link to the first multi-arm PEG comprises a thiosuccinimide group, and the link to the second multi-arm PEG comprises a thionorbornane group; and a portion of the PEG arms of the first multi-arm PEG are linked to the masking peptide through a thiosuccinimide group.

The relative amounts/concentrations of the first multi-arm PEG, second multi-arm PEG, crosslinking peptide, and masking peptide in the anisotropic hydrogel are in accordance with the relative amounts/concentrations of the corresponding reactants as described in the synthesis methods disclosed herein. For example, in some aspects, the amount of PEG arms of the first multi-arm PEG that are linked to the masking peptide, to the mount of PEG arms of the first multi-arm PEG that are linked to the crosslinking peptide in the hydrogel is a ratio of about 1:18 to about 3:9, e.g., about 1:18 to about 2:9, and in one example, about 1:9. In some aspects, the amount of the total thionorbornane linkages, to the amount of the total thiosuccinimide linkages in the hydrogel is a ratio of about 1:20 to about 10:20, e.g., about 1:20 to about 5:20, and in one example, about 3:20.

In some aspects, the anisotropic hydrogel further comprising cells. In some aspects, the cells are from musculoskeletal tissues. In some embodiments, the cells include tenocytes, ligamentocytes, tenoblasts, tendon stem/progenitor cells, osteoblasts, osteocytes, osteoclasts, bone lining cells, chondroblasts, chondrocytes, skeletal stem/progenitor cells, skeletal muscle cells (myocytes), skeletal muscle fibers, satellite cells, myoblasts, myosatellite cells, type A synoviocytes, type B synoviocytes, fibroblasts, mesenchymal stem cells, macrophages, neutrophils, etc., or any combination thereof. In one embodiment, the cells are tenocytes.

In some aspects, the anisotropic hydrogels are synthesized by the methods disclosed herein.

In some aspects, the anisotropic hydrogel is characterized by: (i) a ratio of the elastic modulus of the hydrogel in a direction perpendicular to the applied force, to the elastic modulus of the hydrogel in a direction parallel to the applied force, being about 1:5 to about 2:3, such as about 1:4, 1:3, or 1:2; and/or (ii) a Herman's orientation parameter being about 0.2 to about 0.9, such as about 0.3, 0.4, 0.5, 0.6, 0.7, or 0.8.

Also provided are methods of using the anisotropic hydrogel for tissue (e.g., musculoskeletal tissue) engineering, and/or treating an injury or disease of the musculoskeletal system (e.g., tendon) in a subject.

### EXAMPLES

The following examples are provided to illustrate particular features of certain aspects of the disclosure, but the scope of the claims should not be limited to those features exemplified.

While more than 16 million people sustain tendon injuries annually, few will fully heal, and re-injury rates are high. Limited restoration of function is attributed to fibrosis, characterized by disorganized scar tissue that replaces the anisotropic native tissue. However, fabrication strategies and biomaterials used in prior studies often involve complex processing techniques or the inclusion of additives to induce alignment, limiting tunability of scalable material properties such as modulus or degree of anisotropy. These approaches also limit control of scaffold degradability, a vital component of an effective engineered extracellular matrix (eECM).

To overcome these shortcomings, hydrogels formed via an innovative 2-stage polymerization process were developed and are described herein, which provides anisotropic guidance and biochemical cues in balance with hydrogel remodeling to orchestrate native tendon deposition.

A novel design for anisotropic poly(ethylene glycol) (PEG) hydrogel-based eECM has been developed to promote the alignment of tenocytes. Notably, these PEG-based materials synthesized via two-stage polymerization enable control over polymer architecture and facile integration of biochemical cues for cell adhesion and remodeling. In the first stage, a network is formed via a Michael-addition reaction of multi-arm PEG-maleimide and dicysteine peptides (nondegradable, matrix metalloproteinase (MMP)-degradable, or a combination thereof) and peptides of one cysteine. The network is strained to introduce anisotropy, followed by a secondary thiol-ene photocrosslinking of remaining peptide thiols and multi-arm PEG-norbornene to retain strain-induced alignment. It is shown that anisotropic, cellularly degradable PEG hydrogel eECM induces alignment of tenocytes within the eECM, recapitulating the environment and structure critical for regenerative healing.

The anisotropic eECM will serve as a template for guiding tenocytes into an ordered, native tendon-like orientation and promote regenerative healing in vivo. Following flexor tendon injury, the hydrogel eECM with encapsulated tenocytes will be placed in the suture gap during repair. After 7, 14, or 28 days of healing, the tissue will be harvested from the injury site to analyze cell and matrix orientation and tenogenic gene expression via qPCR and histology. Mechanical properties of repaired tendons will be assessed by testing the range of motion (flexion, gliding resistance) and tensile mechanics. Control experiments will include tendon repairs with no hydrogel or an acellular hydrogel.

The two-stage polymerization strategy disclosed herein enables the encapsulation of tenocytes within the anisotropic polymer without compromising biomaterial tunability. This strategy can be used to promote regenerative healing of the musculoskeletal system, including tendon, muscle, etc., that rely on anisotropic cues to develop functional tissue. Using multiple controlled orthogonal polymerization steps in a PEG-based hydrogel scaffold enables manipulation of biophysical cues via polymer network orientation as well as the incorporation of biochemical cues, such as adhesive epitopes or cellularly degradable crosslinks. This approach also allows for independently controlling and deconvoluting their independent impact on encapsulated tenocyte function. Further, this approach is the first to form a single-network crosslinked biomaterial that does not need to be spun/drawn into fibers or leverage additives to induce anisotropy. This approach offers precise tunability in degradation and anisotropy based on inherent polymer network chemistry. It eliminates diffusion, aggregation, and biocompatibility complications associated with nanoinclusions and enables scaling without compromising material properties. Furthermore, the anisotropy is introduced directly to the polymer chains (rather than via micropatterning or lithography of amorphous/isotropic polymers).

### Example 1

### Materials and Methods

Norbornene-functionalized poly(ethylene glycol) synthesis: Eight-arm norbornene-functionalized poly(ethylene glycol) (PEG-NB) was synthesized via a four step procedure that is detailed in prior work (March, Alyson et al. “Leveraging the predictive power of a 3D in vitro vascularization screening assay for hydrogel-based tissue-engineered periosteum allograft healing,” Biomaterials advances, vol. 169 214187. 15 Jan. 2025, doi: 10.1016/j.bioadv.2025.214187, which is incorporated by reference herein in its entirety). Briefly, eight-arm PEG (20,000 g/mol, tripentaerythritol core, JenKem Technology USA) was functionalized with mesylate end groups (PEG-OMs) via a nucleophilic substitution reaction with methanesulfonyl chloride. PEG-OMs was then functionalized with azide groups (PEG-N₃) via nucleophilic substitution with sodium azide. Next, PEG-N₃ was reduced to amine end group functionalization (PEG-NH₂) via the Staudinger reaction with triphenylphosphine. Finally, PEG-NH₂ was functionalized with norbornene end groups via coupling of 5-norbornene-2-carboxylic acid to the amine groups using a dicyclohexylcarbodiimide (DCC) coupling reaction. PEG-NB was purified by dialysis and subsequent lyophilization.

Peptide synthesis: Peptides, including the dithiol nondegradable peptide GKKCGIQQWGGPCKKG (SEQ ID NO: 21) and MMP degradable peptide GKKCGPQG↓IWGQCKKG (SEQ ID NO: 19) and monothiol adhesive peptide CRGDSG (SEQ ID NO: 11), were synthesized as described in previous work (March et al., Biomaterials Advances, 2025).

Human Tenocyte Isolation: Human tenocytes are isolated from healthy surgically discarded tendon tissue via digestion with Collagenase I. Characterization of tenogenic markers (Scx, Mkx, Tnmd) via qPCR and staining for β-galactosidase are conducted on isolated cells after digestion and again after culture for 3-5 days to ensure minimal senescence and phenotypic drift before encapsulation in hydrogels.

Anisotropic hydrogel synthesis: Anisotropic hydrogels were synthesized using eight-arm PEG-NB, four-arm maleimide-functionalized PEG (20,000 g/mol, PEG-Mal, JenKem Technology USA), cysteine-functionalized adhesive peptide (monothiol peptide), and dicysteine-functionalized MMP-degradable or nondegradable peptide (dithiol peptide). Lithium phenyl-2,4,6-trimethylbenzoylphosphinate (LAP, synthesized as described in Fairbanks, Benjamin D et al. “Photoinitiated polymerization of PEG-diacrylate with lithium phenyl-2,4,6-trimethylbenzoylphosphinate: polymerization rate and cytocompatibility,” Biomaterials vol. 30,35 (2009): 6702-7. doi: 10.1016/j.biomaterials.2009.08.055, which is incorporated by reference herein in its entirety) was used as a photoinitiator. Anisotropic networks were synthesized via a two-step polymerization process. All starting materials were combined in solution with phosphate buffered saline (PBS) at an overall concentration of 5 wt %. PEG-NB, PEG-Mal, and LAP were first combined in solution to ensure homogenous mixing before adding a second solution containing the cysteine-functionalized peptides to initiate the first-stage crosslinking step via a Michael-addition reaction. In the first-stage thiol-Michael reaction, a lightly crosslinked network was formed between the PEG-Mal and dicystene-functionalized peptides. Dithiol peptide and monothiol peptide were added in ratios of 1.05:1 and 0.10:1 thiol: maleimide, respectively, by functional group relative to PEG-Mal (overall thiol: maleimide ratio of 1.15:1). The solution was deposited between two Rain-X coated glass slides separated by 1 mm thick spacers and allowed to react for 20 minutes at room temperature. After 20 minutes, the crosslinked hydrogel was removed from the glass slides and taped to calipers across a 1 mm gap. The gel was stretched uniaxially by sliding the calipers to 0%, 100%, 200%, 300%, 400%, or 500% strain. While retaining the applied strain, the gel was exposed to UV light (365 nm, 5 mW/cm², 5 min) to initiate a second crosslinking step via thiol-ene reaction between the norbornene groups of PEG-NB and pendant thiols on the first-stage network. PEG-NB was included in solution at a ratio of 1:1 thiol: norbornene by functional group relative to the excess thiol (i.e., 0.15:1 norbornene: maleimide, by functional group). LAP was included in solution at a concentration of 0.1 wt % to initiate the photopolymerization. After the second crosslinking stage, the hydrogel was trimmed at the edges of the calipers to remove excess material that was positioned under the tape and not subjected to strain/UV light.

Molar ratios of network components are based on preliminary experiments elucidating the network properties necessary for sufficient deformation of the first-stage network and subsequent retention of applied strain after photopolymerization in the second stage.

To fabricate isotropic control samples, the same two-stage polymerization protocol was followed, but the hydrogel was left on the glass slide with no strain applied for both thiol-Michael and thiol-ene reaction steps.

For materials with encapsulated cells, primary human tenocytes were mixed with the initial reaction solution at a concentration of 1×10⁶ cells/mL. After removing the final material from the calipers, the hydrogels with encapsulated tenocytes were placed in a well plate with fresh media (DMEM/F12 supplemented with 10% FBS and 1% antimycotic/antibiotic).

Characterization of Anisotropic eECM Mechanical Properties: The hydrogel are characterized by tensile testing using dynamic mechanical analysis, analyzing clastic modulus (stress over strain) perpendicular and parallel to the direction of applied strain. The hydrogels will be synthesized to enable clamping and uniform stretching in each direction relative to alignment (e.g., 1 cm width×1 cm length×1 mm thickness). Modulus values are expected to be higher in the direction parallel to the direction of applied strain (the direction in which chains have already been elongated) compared to the perpendicular direction, and mechanical properties are expected to be consistent across compositions. Storage modulus will be quantified using oscillatory shear photorheology during two-stage polymerization in unaligned samples to confirm comparable moduli at each stage and ensure similar reactivities, thus network topologies, using the varied combinations of peptides.

### Example 2

### Characterization of Anisotropy and Mechanical Properties

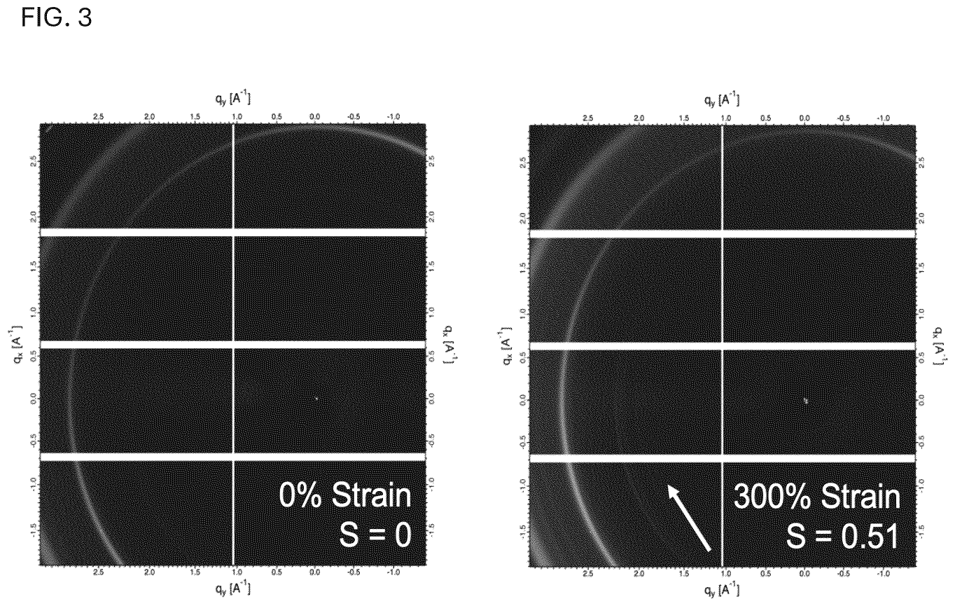

Anisotropic hydrogels were synthesized with a range of strain applied to induce anisotropy. 2D wide angle x-ray scattering (WAXS) diffraction patterns were collected at the BioPACIFIC Materials Innovation Platform facilities at the University of California Santa Barbara. Intensity profiles from 2D diffraction patterns were used to calculate Hermans orientation parameter (Hermans, J. J et al., “Quantitative evaluation of orientation in cellulose fibres from the X-ray fibre diagram,” Recl. Trav. Chim. Pays-Bas, 65:427-447 (1946), https://doi.org/10.1002/recl.19460650605, incorporated by reference in its entirety). With 0% strain, the material remained isotropic as indicated by an order parameter of 0. When strained to 300%, an order parameter of 0.51 is achieved, a value characteristic of highly oriented crosslinked polymer systems (FIG. 3).

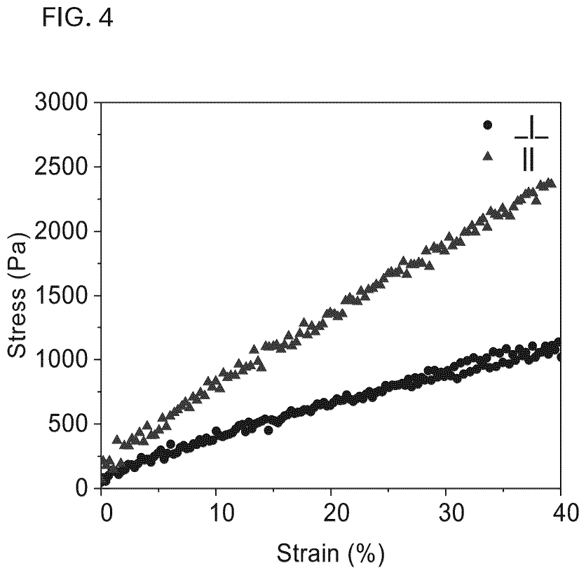

Tensile testing was conducted on anisotropic hydrogels with strain applied along either the axis parallel (triangles) or perpendicular (circles) to the direction of polymer alignment (10% strain/min). Tensile curves plotted (FIG. 4) show that the polymer is ˜2-fold stiffer along the direction of alignment, indicating that the polymer chains are more highly oriented along this direction.

### Example 3

### Alignment of Tenocytes in Anisotropic Hydrogels

To investigate how anisotropy of the hydrogel affects cellular organization within the hydrogels, tenocyte encapsulation experiments were performed. The hydrogels with encapsulated tenocytes were observed with a fluorescence microscope, and the distributions of tenocyte orientation relative to the direction of eECM alignment are plotted.

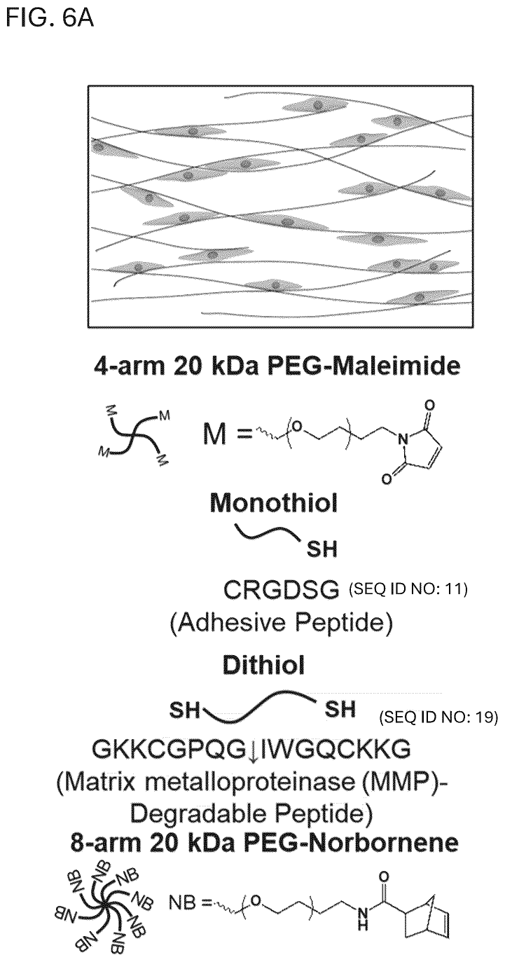

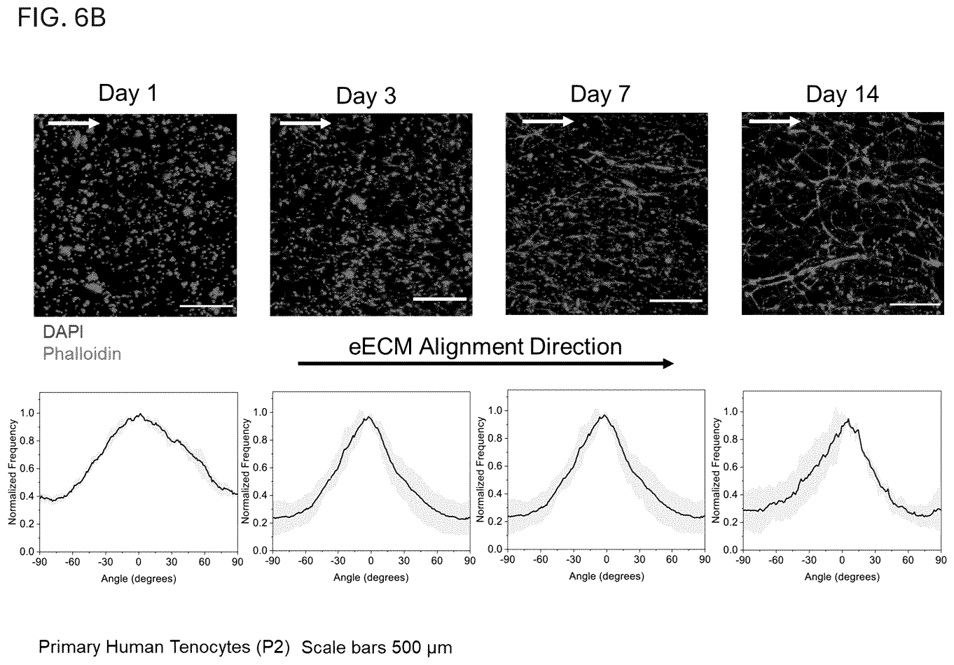

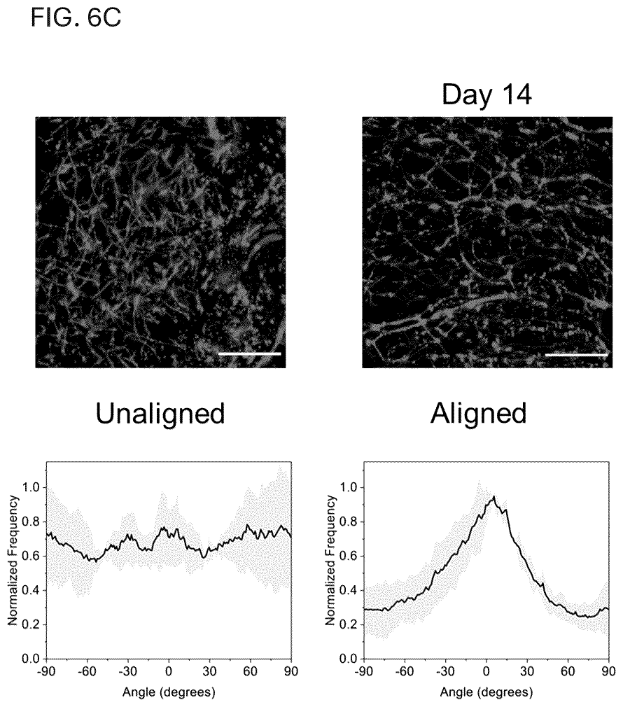

Given that the 300% strain resulted in a highly ordered anisotropic network, tenocytes were encapsulated within the engineered extracellular matrix (eECM) formed from gels strained at 300% (anisotropic) versus 0% as controls at densities consistent with uninjured tendons (1×10⁶ cells/mL). As shown in FIG. 6C, anisotropy is critical to induce tenocyte alignment, as only within the 300% strain gels is there any alignment observed at day 14.

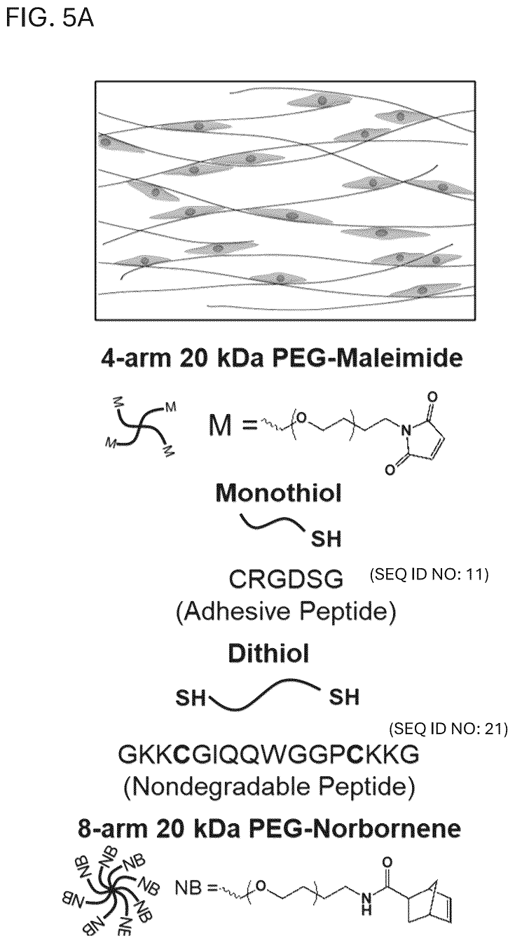

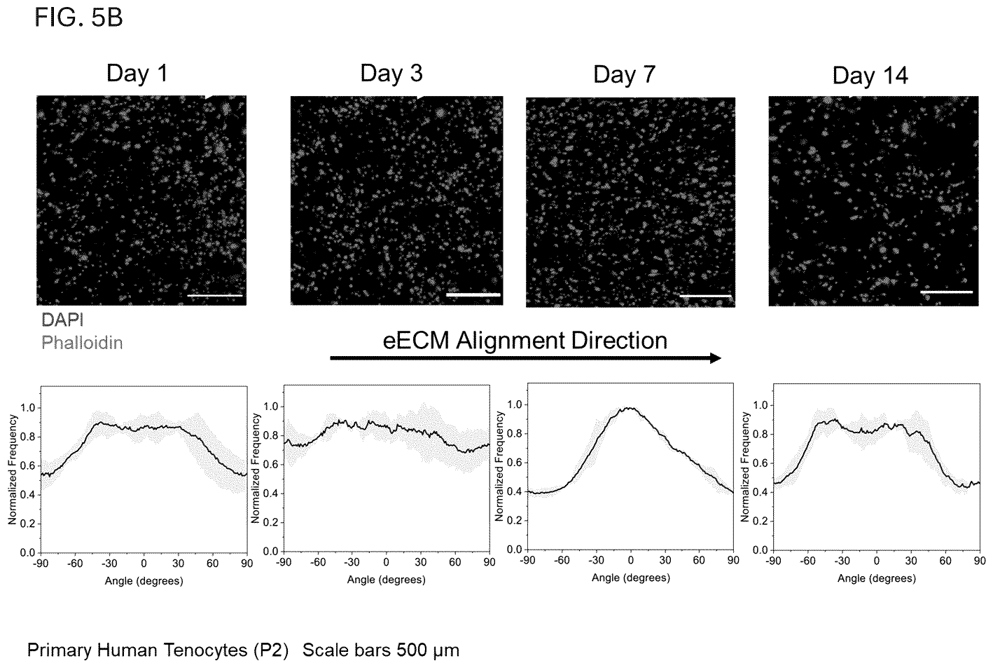

Human tenocytes were encapsulated in eECM subject to either 300% or 0% applied strain, and composed of either nondegradable (FIGS. 5A-5B) or degradable (FIGS. 6A-6C) crosslinks and monitored for 14 days. Tenocytes were encapsulated within anisotropic or isotropic hydrogel eECM formed from peptides at densities consistent with uninjured tendons. Cells (1×10⁶ cells/mL) were encapsulated in a crosslinked network formed via a Michael addition reaction with four-arm PEG-maleimide and a combination of cysteine-functionalized RGD peptide and dicysteine-functionalized nondegradable (FIG. 5A) or MMP-degradable (FIG. 6A) peptide. This network was then stretched (300% strain) to introduce anisotropy. This step was not performed for the isotropic hydrogel control of the same composition. Next, a secondary crosslinking step was performed using thiol-ene photopolymerization of the excess thiols with an eight-armed PEG-norbornene to retain alignment.

For both nondegradable (FIG. 5B) and degradable (FIG. 6B) anisotropic hydrogels, encapsulated tenocytes adopt eECM anisotropy, whereas for either nondegradable (data not shown) or degradable (FIG. 6C) isotropic hydrogels, encapsulated tenocytes do not align. Moreover, in degradable anisotropic hydrogels (FIG. 6B), tenocytes more rapidly align and maintain alignment for the full 14 days of culture while in nondegradable anisotropic hydrogels (FIG. 5B), alignment requires 7 days and is lost after 14 days likely due to lack of matrix remodeling and deposition of native matrix.

Tenocytes encapsulated in the networks with MMP-degradable peptides adopted an elongated morphology along the direction of eECM alignment, with alignment persisting through 14 days, indicating that the anisotropic eECM design successfully provided structural guidance for the cells. Comparatively, the tenocyte morphology changed minimally in nondegradable eECM.

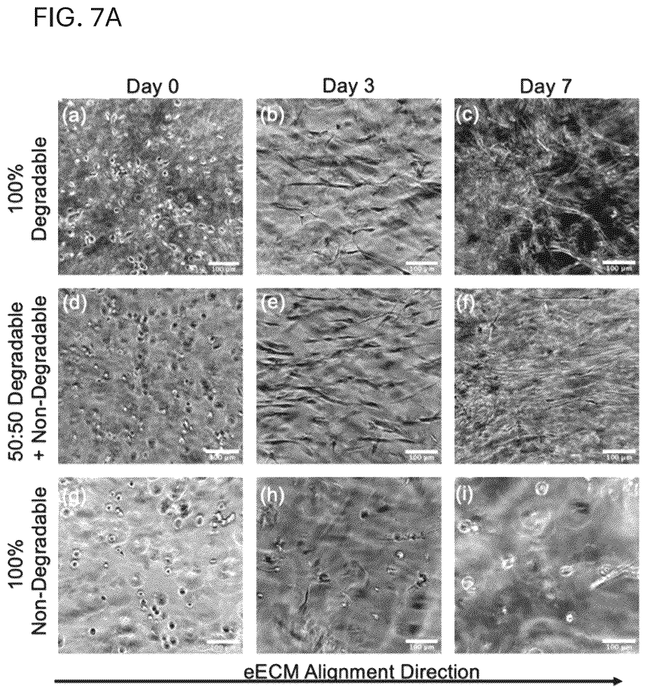

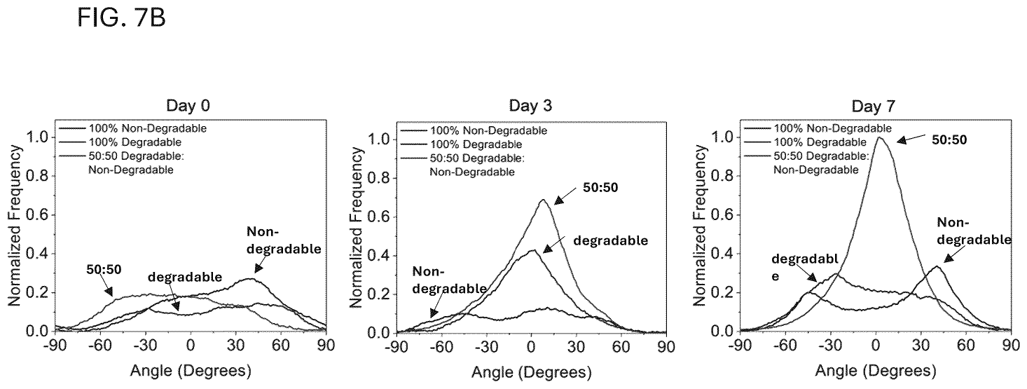

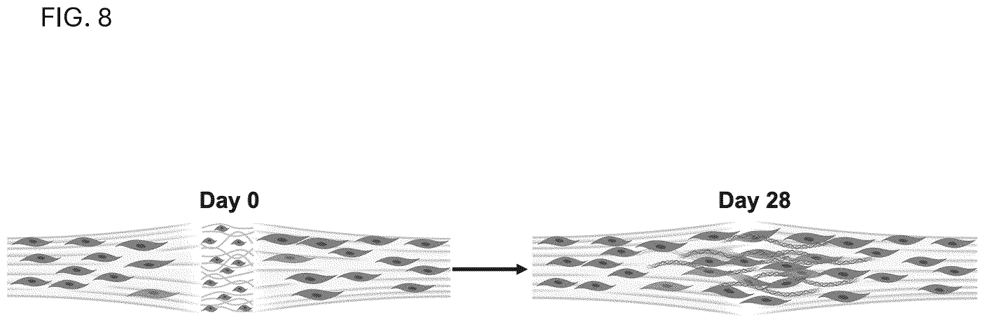

Human tenocytes were encapsulated in three eECM synthesized using MMP-degradable, non-degradable, and equivalent concentrations of MMP-degradable and non-degradable crosslinks. During fabrication, 300% strain was applied to induce anisotropy in the eECM. After three days, tenocytes encapsulated in the networks with either MMP-degradable or equivalent concentrations of MMP-degradable crosslinks and non-degradable crosslinks adopted an elongated morphology along the direction of eECM alignment (FIGS. 7A and 7B), indicating that the anisotropic eECM design successfully provided structural guidance for the cells.

### Example 4

Orchestrating Regenerative Tendon Healing Using Anisotropic eECM In Vivo

Anisotropic hydrogel eECM placed in the suture gap of surgically repaired mouse flexor tendons will induce regenerative healing characterized by anisotropic tissue organization and tenogenic gene expression.

The goal of recapitulating an anisotropic, tenogenic environment in synthetic hydrogel eECM is to promote regenerative healing (rather than fibrotic scarring) of injured tendons. During surgical repair, the tendon is sutured together at the rupture site, and cells bridge the gap between the two sutured ends. When allowed to heal naturally, these cells create new tissue as an organized bridging core surrounded by disorganized scar tissue. Therefore, methods for intervention beyond suturing are needed to create a pro-regenerative environment for these cells and reduce fibrotic scarring after repair.

Anisotropic hydrogel eECM will be implanted during repair to guide cellular alignment across the suture gap as the tendon heals, using a previously developed flexor digitorium longus (FDL) transection model.

Anisotropic hydrogel eECM with encapsulated tenocytes will be used as a template for cellular alignment during tendon healing. The eECM with encapsulated tenocytes will be implanted in the suture gap during repair of ruptured flexor tendons in mice. This includes an anisotropic degradable hydrogel, strained at 300%, and an isotropic, unstrained degradable control. Acellular anisotropic degradable hydrogels, untreated, and sham surgery will be controls for these studies. After 7, 14, or 28 days of healing, the tissue will be harvested from the injury site to analyze cell and matrix orientation and tenogenic gene expression via qPCR and histology. Mechanical properties of repaired tendons will be assessed by testing range of motion (flexion, gliding resistance) and tensile mechanics.

### Synthesis and Implantation of Anisotropic Hydrogels

Synthesis of Anisotropic Hydrogels: The anisotropic hydrogels used in these studies will be synthesized according to the methods described herein. Tenocyte encapsulated hydrogels will be synthesized with the composition including a degradable peptide, and 300% strain will be applied for alignment before implantation. Acellular and isotropic hydrogels will be used as controls.

Acute tendon injury and repair: 16-20-week-old, skeletally mature mice will undergo surgical transection and repair of the flexor digitorum longus (FDL) tendon in the hind paw, as previously described. The B6.Cg-Foxn1nu/J strain (Jackson Laboratory) will be used, as it is athymic, enabling human cell transplantations performed here without immune rejection, and is backcrossed on C57Bl/6 mice, which is typically used for tendon regenerative approaches. The distal FDL tendon will be exposed and transected; anisotropic hydrogel eECM of 1 mm length will be placed between the tendon ends, and two horizontal 8-0 sutures will be placed in the tendon ends. As a control, the tendon will be sutured with no hydrogel in the suture gap to approximate an end-to-end repair. As another control, a sham surgery procedure in which the skin is opened, tendon is exposed but not transected, and the skin is closed with suture. This repair model has been extensively characterized, and experimental findings have been confirmed in samples of human tendon scar tissue that forms following flexor tendon repair surgery, demonstrating the power of this preclinical model to test the efficacy of developed clinically relevant paradigms. While this application uses the FDL tendon as a tendon injury model, the scar tissue process is recapitulated in all tendon and ligament injuries.

### Temporal Analysis of Regenerative Healing

Characterization of Tenocyte Alignment and Tissue Elaboration: Healing tendons will be harvested for paraffin histology and immunofluorescent (IF) analyses at 7-, 14-, and 28-days post-surgery. Time points up to 28 days are chosen for analyses, as recent data show that critical, transient stages of healing occur within this time frame after injury, which encompasses transient myofibroblast and macrophage interactions to facilitate regenerative or fibrotic tissue formation. Serial five-micron paraffin tissue sections will be cut through the sagittal plane of the healing tendon in the intact hind paw. To assess tissue morphology, adjacent sections will be stained with phalloidin and DAPI, and image analysis will be used to quantify the distribution of cellular orientation relative to the direction of network alignment. Overall, changes in the microenvironment consistent with more regenerative tendon healing will be assessed via histology for general tissue deposition and immunofluorescence for collagen I and III and αSMA, a marker for fibrosis. Uninjured tendon and tendon repaired with no eECM will be used as controls.

Analysis of Tenocyte Phenotype: Healing tendons will be harvested for gene expression analyses at 7-, 14-, and 28-days post-surgery. qPCR will be used to identify markers of tenogenesis (Scx, Tnmd, Mkx, αSMA) and collagen types I and III.

Functional Analyses-Tendon Range of Motion: Following harvest, the FDL tendon will be isolated at the myotendinous junction. The proximal tendon will be secured in tape using cyanoacrylate, and a range of weights (0-19 g) will be applied to induce flexion of the digits. The Metatarsophalangeal (MTP) joint angle will be measured to derive two parameters of scar formation: MTP flexion angle, the degree of flexion at 19 g, and Gliding Resistance, a measure of the ROM over the applied loads. A lower MTP flexion angle and higher Gliding Resistance indicates increased scar tissue formation and impaired gliding function.

Functional Analyses Assessment of structural and material properties: Changes in mechanical properties will be assessed via endpoint measurements from 7, 14, and 28 days post-surgery. Changes in the structural and material properties will be assessed via tensile testing following isolation of the healing FDL after ROM testing. The FDL will be tested in tension until failure, force-displacement, and stress-strain data will be plotted and analyzed for structural and material properties. Images acquired from orthogonal orientations will be used to determine specimen gauge length and cross-sectional area.

### Statistical Analyses and Consideration of Biological Variables

For quantitative measures, statistical comparisons between groups will use analysis of variance (ANOVA) with post-hoc comparisons using Fisher's Least Square Differences and Bonferroni correction. All material compositions will be synthesized and tested in triplicate (e.g., independent cell isolations), with 5 technical replicates per isolation. Significance is p<0.05 for all comparisons. Sample sizes were determined based on post-hoc power analyses of our published and preliminary data. While it is unclear whether sexual dimorphism is relevant to in vitro tenocytes, sample sizes are powered to detect sex differences, and data will be analyzed as a composite and stratified by sex and age.

We claim:

1. A method of synthesizing an anisotropic hydrogel, comprising:
reacting a multi-arm poly(ethylene glycol) (PEG) comprising maleimide end groups (PEG-maleimide), with a dithiol crosslinker and a monothiol molecule, thereby generating a first-stage crosslinked network with pendant thiol groups;
applying mechanical force to stretch the first-stage crosslinked network to 50-500% strain;
reacting the pendant thiol groups with a multi-arm PEG comprising norbornene end groups (PEG-norbornene), by subjecting the first-stage crosslinked network to UV light in the presence of a photoinitiator, thereby generating the anisotropic hydrogel.

2. The method of claim 1, wherein
the molar amount of the maleimide end groups to the molar amount of thiol groups in the dithiol crosslinker and the monothiol molecule is a ratio of about 20:23 (maleimide:thiol),
the molar amount of monothiol molecule to the molar amount of dithiol crosslinker is a ratio of about 4:21 (monothiol molecule: dithiol crosslinker), and/or
the molar amount of the pendant thiol groups to the molar amount of norbornene end groups is a ratio of about 1:1.

3. The method of claim 2, wherein
the multi-arm PEG-maleimide comprises 3-arm, 4-arm, 6-arm, 8-arm, or 10-arm PEG, or any combination thereof;
the multi-arm PEG-norbornene comprises 3-arm, 4-arm, 6-arm, 8-arm, or 10-arm PEG, or any combination thereof;
the multi-arm PEG-maleimide has a molecular weight of about 2 to about 40 kDa, or any combination thereof; and/or
the multi-arm PEG-norbornene has a molecular weight of about 2 to about 40 kDa, or any combination thereof.

4. The method of claim 1, wherein
the dithiol crosslinker comprises a peptide having two cysteines;
the monothiol molecule comprises a peptide having one cysteine;
the dithiol crosslinker comprises a peptide that is 4-100 amino acids in length, or a combination thereof; and/or
the monothiol molecule comprises a peptide that is 4-100 amino acids in length, or a combination thereof.

5. The method of claim 1, wherein
the dithiol crosslinker comprises a peptide comprising a matrix metalloproteinase (MMP)-degradable sequence, and/or
the monothiol molecule comprises an adhesive peptide.

6. The method of claim 5, wherein
the matrix metalloproteinase (MMP)-degradable sequence is GPQGIWGQ (SEQ ID NO: 12), and/or
the adhesive peptide is CRGDSG (SEQ ID NO: 11).

7. The method of claim 1,
wherein the multi-arm PEG-maleimide, the multi-arm PEG-norbornene, the dithiol crosslinker, the monothiol molecule, and the photoinitiator are present together in a mixture.

8. The method of claim 7, wherein the concentration of the photoinitiator (wt %) to the total concentration (wt %) of the multi-arm PEG-maleimide, the multi-arm PEG-norbornene, the dithiol crosslinker, the monothiol molecule, and the photoinitiator in the mixture is a ratio of about 1:20 to about 1:80.

9. The method of claim 7, wherein the mixture further comprises cells.

10. The method of claim 9, wherein the cells are from musculoskeletal tissues.

11. An anisotropic hydrogel generated by the method of claim 1.

12. The anisotropic hydrogel of claim 11, wherein
the ratio of the elastic modulus of the hydrogel in a direction perpendicular to the applied force, to the elastic modulus of the hydrogel in a direction parallel to the applied force, is about 1:5 to about 2:3; and/or
the hydrogel exhibits a Herman's orientation parameter of about 0.2 to about 0.9.

13. An anisotropic hydrogel comprising a cross-linked network of a first multi-arm poly(ethylene glycol) (PEG), a second multi-arm PEG, a crosslinking peptide, and a masking peptide, wherein
the crosslinking peptide links together two of the first multi-arm PEG, or links together one of the first multi-arm PEG and one of the second multi-arm PEG, wherein the link to the first multi-arm PEG comprises a thiosuccinimide group, and the link to the second multi-arm PEG comprises a thionorbornane group; and a portion of the PEG arms of the first multi-arm PEG are linked to the masking peptide through a thiosuccinimide group.

14. The anisotropic hydrogel of claim 13, wherein
the molar amount of PEG arms of the first multi-arm PEG that are linked to the masking peptide, to the molar amount of PEG arms of the first multi-arm PEG that are linked to the crosslinking peptide is a ratio of about 1:9; and/or
the molar amount of the total thionorbornane linkage, to the molar amount of the total thiosuccinimide linkage, is a ratio of about 3:20.

15. The anisotropic hydrogel of claim 14, wherein
the first multi-arm PEG comprises 3-arm, 4-arm, 6-arm, 8-arm, or 10-arm PEG, or any combination thereof;
the second multi-arm PEG comprises 3-arm, 4-arm, 6-arm, 8-arm, or 10-arm PEG, or any combination thereof;
the first multi-arm PEG has a molecular weight of about 2 to about 40 kDa, or any combination thereof; and/or
the second multi-arm PEG has a molecular weight of about 2 to about 40 kDa, or any combination thereof.

16. The anisotropic hydrogel of claim 14, wherein
the crosslinking peptide comprises a peptide having two cysteines;
the masking peptide comprises a peptide having one cysteine;
the crosslinking peptide comprises a peptide that is 10-20 amino acid in length, or a combination thereof; and/or
the masking peptide comprises a peptide that is 4-10 amino acid in length, or a combination thereof.

17. The anisotropic hydrogel of claim 13, wherein
the crosslinking peptide comprises a matrix metalloproteinase (MMP)-degradable sequence, and/or
the masking peptide comprises an adhesive peptide.

18. The anisotropic hydrogel of claim 13, further comprising cells.

19. The anisotropic hydrogel of claim 13, wherein
the ratio of the elastic modulus of the hydrogel in a direction perpendicular to the applied force, to the elastic modulus of the hydrogel in a direction parallel to the applied force, is about 1:5 to about 2:3; and/or
the hydrogel exhibits a Herman's orientation parameter of about 0.2 to about 0.9.

20. A composition for generating an anisotropic hydrogel, comprising a multi-arm poly(ethylene glycol) (PEG) comprising maleimide end groups (PEG-maleimide),
a multi-arm PEG comprising norbornene end groups (PEG-norbornene),
a dithiol crosslinker, and
a monothiol molecule,
wherein the molar amount of the maleimide end groups to the molar amount of thiol groups in the dithiol crosslinker and the monothiol molecule is a ratio of about 20:23 (maleimide:thiol),
the molar amount of monothiol molecule to the molar amount of dithiol crosslinker is a ratio of about 4:21 (monothiol molecule: dithiol crosslinker), and
the molar amount of the norbornene end groups to the molar amount of the maleimide end groups is a ratio of about 3:20.
